(19) United States
(12) Patent Application Publication
(10) Pub. No.: US 2023/0136997 A1
(43) Pub. Date: May 4, 2023

(54) TARGETING ABCB5 IN GLIOBLASTOMA MULTIFORME

(71) Applicants: Children's Medical Center Corporation, Boston, MA (US); The Brigham and Woman's Hospital, Inc., Boston, MA (US)

(72) Inventors: Markus H. Frank, Cambridge, MA (US); Natasha Y. Frank, Cambridge, MA (US)

(73) Assignees: Children's Medical Center Corporation, Boston, MA (US); The Brigham and Woman's Hospital, Inc., Boston, MA (US)

(21) Appl. No.: 17/918,479

(22) PCT Filed: Apr. 14, 2021
(86) PCT No.: PCT/US2021/027356

§ 371 Date: Oct. 12, 2022

Related U.S. Application Data
(60) Provisional application No. 63/010,643, filed on Apr. 15, 2020.

(51) Int. Cl. A61K 39/395; A61K 31/495; C12Q 1/6886; C07K 16/28; C07K 16/40; A61P 35/00; A61P 25/00; C07K 16/30

(52) U.S. Cl. CPC A61K 39/3955; A61K 31/495; C12Q 1/6886; C07K 16/28; C07K 16/40; A61P 35/00; A61P 25/00; C07K 16/3053; C07K 2317/31; C07K 2317/622; A61K 2039/505

(57) ABSTRACT

The present invention is directed to methods and compositions for treating Glioblastoma multiforme (GBM). The risk of developing therapy resistant GBM may be assessed by detecting the presence of ABCB5 in the GBM cells. Therapeutic interventions utilizing an ABCB5 blockade to sensitize the GBM cells to therapeutic agents such as temozolomide are provided.

### RELATED APPLICATIONS

This application claims priority under 35 U.S.C. § 119(e) to U.S. Provisional Patent Applications, U.S. Ser. No. 63/010,643, filed Apr. 15, 2020, the entire contents of which is incorporated herein by reference.

### FEDERALLY SPONSORED RESEARCH

This invention was made with government support under 5 R01EY025794 05, awarded by the NIH/NEI, and 5 I01 BX000516 08, awarded by Veterans Affairs. The government has certain rights in the invention.

### BACKGROUND OF INVENTION

Glioblastoma multiforme (GBM), the most common primary malignant brain tumor in adults, is associated with poor prognosis and high lethality. Patient survival remains dismal owing to therapeutic resistance to clinically approved drugs such as temozolomide (TMZ) and bevacizumab, and a high rate of tumor recurrence. Due to minimal beneficial effects of existing treatment regimens, there is an urgent need for novel therapeutic strategies targeting therapy-resistant cancer subpopulations, which may correspond to GBM cancer stem cells (CSCs).

TMZ is a clinically approved drug for the treatment of GBM, which leads to significant prolongation of patient survival (Stupp, R., et al., (2005). N Engl J Med 352, 987-996). Although acting as a cytotoxic imidazotetrazine, TMZ can induce a G2/M arrest (Newlands, E. S., et al., (1997). Cancer Treat Rev 23, 35-61, Filippi-Chiela, E. C., et al., (2013). BMC Cancer 13, 147), which allows therapy-resistant cancer cells to repair the DNA before entering into the mitotic or M phase, hence protecting the cells and their progeny from drug-induced cytotoxicity (DiPaola, R. S. (2002). Clin. cancer res., 8: 3512-3519, 2002. Clin Cancer Res 8, 3311-3314, Sherry, C. J. (2000). Cancer Res 60, 3689-3695, Schwartz, G. K., and Shah, M. A. (2005). J Clin Oncol 23, 9408-9421). An essential step for G2/M transition is activation of the Cyclin B1/CDK1 complex. In resting cells, the tyrosine kinases WEE1 and MYT1 induce inhibitory phosphorylation of CDK1, thus maintaining the Cyclin B1/CDK1 complex in an inactive state. As the cells prepare to divide, polo like kinase (PLK1), a major positive regulator of G2/M transition (van Vugt, M. A., and Medema, R. H. (2005). Oncogene 24, 2844-2859), is activated. Subsequently, PLK1 induces expression of CDK1 by inhibiting WEE1 and MYT1. PLK1 also activates CDC25C which in turn plays a pivotal role in dephosphorylation and activation of CDK1. Chemotherapeutic drugs and other DNA damaging agents can severely impair this pathway by activating the sensory ATM/ATR kinases, which phosphorylate and activate CHEK1. CHEK1 phosphorylates and inactivates CDC25C, hence retaining the Cyclin B1/CDK1 complex in an inactive phosphorylated state and eventually causing G2/M arrest.

### SUMMARY OF INVENTION

The role of ABCB5 in growth and chemoresistance of Glioblastoma multiforme (GBM) has been examined herein. It was found that ABCB5 is expressed in primary GBM tumors, and that expression was significantly correlated with overall poor survival. Moreover, ABCB5 was also expressed by CD133-positive CSCs in the established human U-87 MG, LN-18, and LN-229 GBM cell lines. Antibody- or shRNA-mediated functional ABCB5 blockade inhibited proliferation and survival of GBM cells and sensitized them to temozolomide (TMZ)-induced apoptosis in vitro. Likewise, in in vivo human GBM xenograft experiments with immunodeficient mice, monoclonal antibody treatment inhibited growth of mutant TP53, wild-type PTEN LN229 tumors and sensitized LN229 tumors to TMZ therapy. It was demonstrated that ABCB5 blockade inhibits TMZ-induced G2/M arrest and augments TMZ-mediated cell death. Thus, the data disclosed herein demonstrate that ABCB5 is a GBM chemoresistance marker and that targeting ABCB5 is useful for improving GBM therapies.

The present invention, in some aspects, is directed to methods and compositions for modulating ABCB5+ cancer stem cell activity.

Aspects of the invention relate to a method for treating Glioblastoma multiforme (GBM), by administering to a subject having GBM an inhibitor of ATP-binding cassette subfamily B member 5 (ABCB5) and a chemotherapeutic agent, wherein the chemotherapeutic agent is an alkylating agent in an effective amount to treat the GBM. In some embodiments, the alkylating agent is Temozolomide.

A method for treating a chemoresistant cancer is provided in other aspects. The method involves identifying a subject having a chemoresistant cancer, administering an effective amount of an ABCB5 inhibitor to reverse a chemotherapy induced G2/M arrest in cancer cells of the subject and administering a chemotherapeutic agent to the subject to promote cancer cell death.

A method for treating a cancer in a subject, wherein the cancer is associated with G2/M cell cycle arrest by identifying the cancer as a cancer having cells in G2/M cell cycle arrest and administering an effective amount of an ABCB5 inhibitor to the subject to reverse G2/M cell cycle arrest in the cells is provided in other aspects.

In some embodiments the subject is further administered a chemotherapeutic agent to promote cancer cell death.

In some embodiments, the chemotherapeutic agent is an alkylating agent. In some embodiments, the alkylating agent is Temozolomide.

In some embodiments, the subject is identified as having an ABCB5+ cancer.

In some embodiments, the inhibitor of ABCB5 is an anti-ABCB5 antibody or fragment thereof. In some embodiments, the anti-ABCB5 antibody or fragment thereof has specificity for a cyclical form or a linear form of an extracellular polypeptide of the protein. In other embodiments, the anti-ABCB5 antibody or fragment thereof alters the conformation of an ABCB5 PIP2 binding site. In some embodiments, the anti-ABCB5 antibody is a monoclonal antibody. In some embodiments the antibody is a bispecific antibody that is specific for ABCB5 and RTKs.

In some embodiments, the inhibitor of ABCB5 is a PIP2 antagonist.

In some embodiments, the inhibitor of ABCB5 is a lipid analog.

In some embodiments, the inhibitor of ABCB5 is an inhibitory nucleic acid.

In some embodiments, the inhibitor of ABCB5 is an enzyme.

In some embodiments, the inhibitor of ABCB5 is anti-receptor tyrosine kinase (RTK) antibody.

In some embodiments, the inhibitor of ABCB5 is an inhibitor of insulin receptor (InsR).

In some embodiments, the inhibitor of ABCB5 is an inhibitor of insulin-like growth factor-1 receptor (IGF1R).

In some embodiments, the inhibitor of ABCB5 is an inhibitor of signal integrating adapter molecule (IRS-1).

In other aspects the invention is a method for identifying a Glioblastoma multiforme (GBM) as a chemoresistant cancer, by isolating a GBM cancer cell from a subject and performing an assay to determine whether the GBM cancer cell expresses ABCB5, wherein if the GBM cancer cell expresses ABCB5 the GBM is a chemoresistant cancer.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1G. ABCB5 expression in human GBM. 1A. Bar graphs depicting copy number alterations as percentage of total cases for GBM (n=146) and LGG (oligodendroglioma: n=189, oligoastrocytoma: n=129, astrocytoma: n=194) brain tumors from TCGA. 1B. Box and whisker plot overlaid with individual data points showing the distribution of mRNA expression (log₂) of ABCB5 in GBM (n=152) and LGG (oligodendroglioma: n=191, oligoastrocytoma: n=130, astrocytoma: n=194) brain tumors from TCGA. Data was analyzed using a Kruskal-Wallis test with Dunn's multiple comparisons test (****P<0.0001). Boxes extend from 1^{st} quartile to 3^{rd} quartile; median indicated by solid line. 1C. Box and whisker plot (left) overlaid with individual data points showing the distribution of mRNA expression (log₂) of ABCB5 in the three GBM subtypes (classical: n=59; mesenchymal: n=51; pronounal: n=46). Boxes extend from 1^{st} quartile to 3^{rd} quartile; median indicated by solid line. Kaplan-Meier plots (right) depicting OS for all GBM subtypes together (n=155, high events=58, low events=65) and each subtype separately (classical: n=59, high events=24, low events=24; mesenchymal: n=51, high events=20, low events=19; perineural: n=66, high events=17, low events=19). OS expressed in percentage and time in months. 1D. Representative immunohistochemical staining for hematoxylin and eosin (H&E) (left) and ABCB5 (right) in clinical GBM. 1E. qRT-PCR of ABCB5 expression from GBM cell lines. Error bars indicate SD. 1F. Nested PCR analysis of ABCB5 mRNA expression in GBM cell lines. G3361, a melanoma cell line, was used as a positive control. Water was used as a negative control. 1G. Representative flow cytometric analyses of ABCB5 expression (FITC, FL1 fluorescence) on human GBM cell lines.

FIGS. 2A-2C. CD133-positive GBM stem cells express ABCB5. 2A. Box and whisker plot overlaid with individual data points showing the distribution of mRNA expression (log₂) of CD133 in GBM (n=152) and LGG (oligodendroglioma: n=191, oligoastrocytoma: n=130, astrocytoma: n=194) brain tumors from TCGA. Data was analyzed using a Kruskal-Wallis test with Dunn's multiple comparisons test (**P<0.01, ****P<0.0001). Boxes extend from 1^{st} quartile to 3^{rd} quartile; median indicated by solid line. 2B. Representative dual-color flow cytometric analysis of ABCB5 (FITC, FL1 fluorescence) and CD133 (APC, FL4 fluorescence) co-expression on human GBM cells. Cells co-expressing ABCB5 and CD133 are found in the upper right quadrant of each fluorescence plot. Data shown are representative of n=3 independent experiments. 2C. Expression of CD133 (APC, FL4 fluorescence) as determined by flow cytometry for LN-229 and LN-18 GBM cell lines. Bar graphs depict fold change in CD133 positivity relative to untreated control cells. Data analyzed using unpaired t-test with Welch's correction. Error bars indicate SEM (n=6, ****P<0.0001).

FIGS. 3A-3E. Antibody-mediated ABCB5 blockade inhibits proliferation and induces apoptosis of GBM cells. 3A. Cell proliferation analyzed by MTT assay (n=9). LN-229, LN-18, and U-87 MG GBM cells were incubated with 0-200 μg/ml ABCB5 mAb or isotype control mAb for 72 hours. Statistical significance was determined using two-way ANOVA with Sidak's multiple comparisons test (*P<0.05, **P<0.01, ****P<0.0001). Error bars indicate SEM. 3B. Representative plot for the percentage of apoptotic cells as determined by dual color flow cytometry for LN-229 (left). Cells in early and late apoptotic stages are found in the lower right and upper right quadrant of each fluorescence plot (respectively). Bar graphs show combined data for LN-229, LN-18, and U-87 MG cell lines (right). Data analyzed using unpaired t-test. Error bars indicate SEM (**P<0.01) (n=6). 3C. In vivo tumor growth kinetics (left) of ABCB5 mAb-vs. isotype control mAb-treated LN-229 and U-87 MG GBM xenografts (n=5). Statistical significance was determined using unpaired t-test (**P<0.01, ns=not significant). Error bars indicate SEM. Bar graphs (right) depict mean tumor weight of anti-ABCB5 mAb- vs. isotype control mAb-treated LN-229 and U-87 MG GBM xenografts (n=5), analyzed using unpaired t-test. Error bars indicate SEM (*P<0.05, ns=not significant). 3D. Representative immunohistochemical staining for Ki-67 and cleaved caspase-3 expression in anti-ABCB5 mAb or isotype control mAb-treated LN-229 GBM xenografts. 3E. Bar graphs represent the percentage of Ki-67-(top) or cleaved caspase-3-(bottom) positive nuclei in anti-ABCB5 mAb-vs. isotype control mAb-treated GBM tumor xenografts. Data analyzed using unpaired t-test. Error bars indicate SEM (**P<0.01, ***P<0.001) (n=5).

FIGS. 4A-4E. Augmentation of TMZ-induced growth inhibitory effects in GBM by antibody-mediated ABCB5 blockade. 4A. Cell proliferation analyzed by MTT assay of LN-229, LN-18, and U-87 MG human GBM cells pre-incubated for 2 hours with 100 μg/ml ABCB5 mAb or isotype control mAb followed by treatment with TMZ (0-1000 μM) for 72 hours (n=9). Error bars indicate SEM (*P<0.05, ****P<0.0001). Statistical significance was determined using two-way ANOVA with Sidak's multiple comparisons test. Error bars indicate SEM. 4B. Percentage of apoptotic cells as determined by dual color flow cytometry for LN229. Cells in early and late apoptotic stages are found in the lower right and upper right quadrant of each fluorescence plot (respectively) (n=3). 4C. Bar graphs represent mean LN-229 or U-87 MG tumor volume of groups treated with TMZ in the presence of ABCB5 mAb or isotype control mAb. Data analyzed by unpaired t-test (isotype control: LN229 n=6, U-87 MG n=4; ABCB5 mAb: LN229 n=6, U-87 MG n=5). Error bars indicate SEM (*P<0.05, ns=not significant). 4D. Representative immunohistochemical staining for Ki-67 and cleaved caspase-3 expression in LN-229 GBM xenografts treated with isotype control mAb, ABCB5 vehicle control, or TMZ in the presence of isotype control mAb or ABCB5 mAb. 4E. Bar graphs represent the mean percentage of Ki-67 and cleaved caspase-3 positive nuclei of groups treated with TMZ in the presence of ABCB5 mAb or isotype control mAb. Data analyzed using unpaired t-test (n=5). Error bars indicate SEM (*P<0.05, **P<0.01).

FIGS. 5A-5C. Enrichment of cell-cycle-related transcripts in ABCB5-positive GBM cells. 5A. PCA of all genes detected by microarray analysis of FACS-sorted ABCB5-positive and ABCB5-negative GBM cells (n=3). 5B. Percentage of categories with key words of interest out of 489 total diseases and functions categories. 5C. Diseases and functions determined by IPA to be enriched between ABCB5-positive and ABCB5-negative GBM cells (n=3). The p-value for a given annotation is calculated by Fishers Exact Test using the number of focus genes that participate in that process in relation to the total number of genes associated with that process in the IPA knowledge base. Genes identified by our study listed to the right.

FIGS. 6A-6D. Antibody-mediated ABCB5 blockade releases GBM cells from TMZ-induced G2/M arrest. 6A. Representative flow cytometric DNA content (propidium iodide, FL2 fluorescence) analysis in LN-229 cells following FL2H versus FL2W analysis for doublet elimination. 6B. Bar graphs represent percentage of cells in G2/M arrest from flow cytometry analysis of LN-229, U-87 and LN-18 GBM cells (n=8). Error bars indicate SEM (*P<0.05). 6C. Western blot analysis of cell cycle checkpoint molecules in LN-229 cells. Molecular weight indicated to left (kDa) 6D. Western blot analysis of cell cycle checkpoint molecules in xenograft tumors from mice treated with TMZ in the presence of anti-ABCB5 mAb or isotype control mAb (n=3). Molecular weight indicated to left (kDa).

FIGS. 7A-7E. Knockdown of ABCB5 mimics growth-inhibition of ABCB5 blockade and releases GBM cells from TMZ-induced G2/M arrest. 7A. IP-Western blot of LN-229 and U-87 MG GBM cells after short hairpin knockdown (KD). 5 mg sonicated and precleared protein loaded per lane. Molecular weight indicated to left (kDa). 7B. Cell proliferation analyzed by MTT assay (n=6). Bar graph represents OD₅₇₀ measurements of LN-229 and U-87 MG GBM cells. Data analyzed using unpaired t-test. Error bars indicate SEM (*P<0.05) 7C. Cell proliferation analyzed by MTT assay of ABCB5 KD LN-229 or U-87 MG human GBM cells and their corresponding Control KD cell variants after treatment with varying concentrations of TMZ (0-1000 μM) for 72 hours. Statistical significance was determined using two-way ANOVA followed by Sidak's multiple comparisons test (**P<0.01, ***P<0.001, ****P<0.0001) (n=7). Error bars indicate SEM. 7D. Flow cytometric DNA content (propidium iodide, FL2 fluorescence) analysis in LN-229 cells following FL2H versus FL2W analysis for doublet elimination. 7E. Wes analysis of cell cycle checkpoint molecules in LN-229 and U-87 MG GBM cells. Lower band depicts loading control (ACTB) with the exception of p-CDC2 (Tyr15) and CDC2 in which ACTB is the upper band. Molecular weight indicated to left (kDa).

### DETAILED DESCRIPTION OF INVENTION

Glioblastoma multiforme (GBM) is a malignant brain tumor with a poor prognosis resulting from tumor resistance to anticancer therapy and a high recurrence rate. A role of ABCB5 in growth and chemoresistance of GBM has been elucidated herein. It was found that ABCB5 is expressed in primary GBM tumors, in which its expression was significantly correlated with overall poor survival. Functional ABCB5 blockade, i.e. by antibody- or inhibitory nucleic acids, inhibited proliferation and survival of GBM cells and sensitized them to anti-cancer drugs such as temozolomide (TMZ)-induced apoptosis. In vivo human GBM xenograft experiments with immunodeficient mice, demonstrated that monoclonal antibody treatment inhibited growth of mutant TP53, wild-type PTEN LN229 tumors and sensitized LN229 tumors to cancer therapy such as TMZ.

It has been demonstrated herein that ABCB5 blockade inhibits drug-induced G2/M arrest in resistant cancer cells and thereby augments drug-mediated cell death. Thus, ABCB5 is a GBM chemoresistance marker as well as a therapeutic target for improving current cancer therapies. In instances when cancer cells arrest in the G2/M cell cycle phase, the cells are able to undergo DNA repair mechanisms and develop resistance to drugs. As demonstrated herein, ABCB5 plays a critical role as a cell cycle regulator. ABCB5 blockade mobilizes resistant tumor cells from the G2/M cell cycle arrest, preventing them from otherwise undergoing repair of their DNA. Therefore, ABCB5 blockade inhibits G2/M cell cycle-associated DNA repair as a novel mechanism to induce tumor cell death, which is shown herein confers therapeutic advantage in the treatment of cancer such as GBM In addition to direct ABCB5 blockade by antibodies, pharmacological agents that block ABCB5, as well as blockers of certain receptor tyrosine kinases (RTKs) and associated signal adapters, for which ABCB5 is important in signal transduction will function according to these methods. Exemplary blockers in this regard would be a blocker of the signaling adapter is Insulin receptor substrate 1 (IRS-1), which integrates signals of the RTKs insulin receptor (InsR) or insulin-like growth factor-1 receptor (IGF1R), or blockers of InsR or IGF1R themselves, for which ABCB5 is required for signal transduction. Such blockers produce inhibition resulting in blockade of ATM/ATR and CHK1/CHK2, leading to p-PLK1 S137/T210 increases and thereby to progression from the G2/M phase.

Tyrosine autophosphorylation of receptor tyrosine kinases (RTKs) plays a critical role in the regulation of kinase activity and in the recruitment and activation of intracellular signaling pathways. Autophosphorylation is mediated by a sequential and precisely ordered intermolecular (trans) reaction. Pathological loss of function mutations or oncogenic activating mutations in the asymmetric contact interface of receptor tyrosine kinases may hinder or facilitate asymmetric dimer formation and trans autophosphorylation, respectively. In one embodiment, the RTK is a fibroblast growth factor receptor (FGFR), e.g., fibroblast growth factor receptor 1 (FGFR1), fibroblast growth factor receptor 2 (FGFR2), fibroblast growth factor receptor 3 (FGFR3), or fibroblast growth factor receptor 4 (FGFR4).

ABCB5 has been established as a key mediator of tumor growth, aggressiveness, and multidrug resistance (MDR) in malignant melanoma, colorectal cancer, hepatocellular, oral squamous and Merkel cell carcinomas, and ocular surface squamous neoplasia. It's role in tumorogencity in other cancers is unknown. Quite surprisingly it has been discovered that ABCB5 plays a unique role in modulating G2/M checkpoint regulation that leads to drug resistance in cancer and that disruption of ABCB5 signaling is sufficient to reverse drug resistance. This is particularly notable in TMZ-induced G2/M arrest in GBM. The ability to rapidly identify TMZ resistant cancers and reverse them presents a significant advance in the treatment of this difficult to treat cancer.

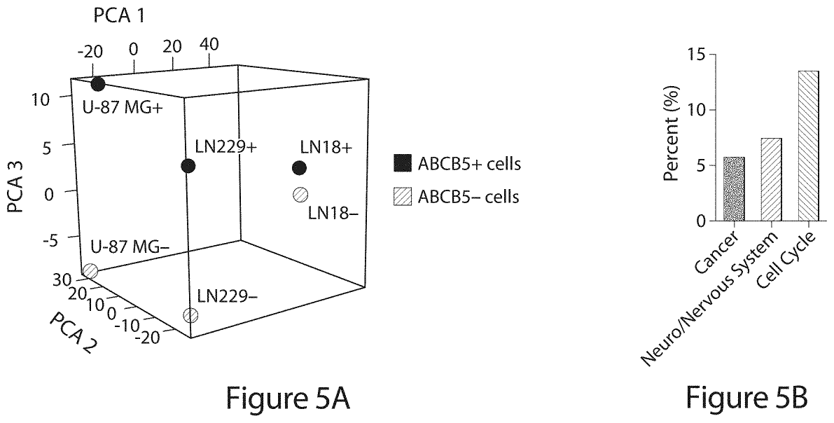

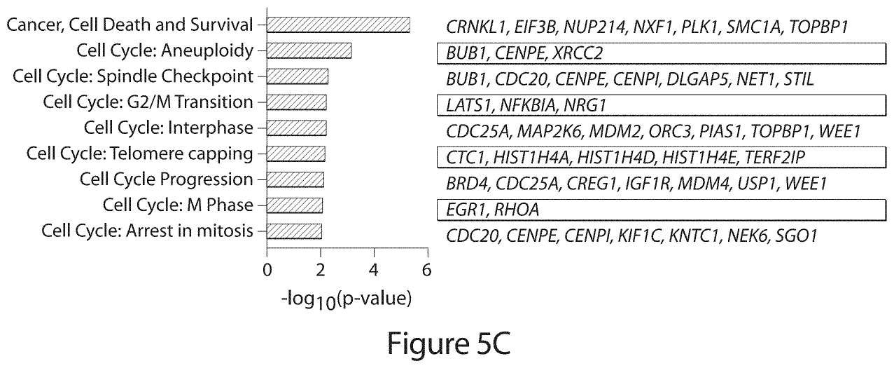

While TMZ remains one of the main therapeutic agents in GBM, less than 50% of patients respond to TMZ therapy (Woo, J. Y., et al., (2015) Continuous Low-Dose Temozolomide Chemotherapy and Microvessel Density in Recurrent Glioblastoma. J Korean Neurosurg Soc 58, 426-431). It has been established that in GBM, TMZ can induce G2/M arrest through activation of ATM/ATR-Chk1/2, which is a pro-survival mechanism that enables cancer cells to repair their DNA prior to mitosis entry. Inhibition of the cell-cycle arrest may result in mitotic catastrophe and cell death (Hirose, Y., et al., (2001) Abrogation of the Chk1-mediated G(2) checkpoint pathway potentiates temozolomide-induced toxicity in a p53-independent manner in human glioblastoma cells. Cancer Res 61, 5843-5849). Specific enrichment of cell cycle regulation-related gene categories such as Cell Cycle: Aneuploidy (3.22), Cell Cycle: Spindle Checkpoint (2.36), and Cell Cycle: G2/M Transition (2.28) was discovered in ABCB5-positive GBM cells (Examples, FIG. 5B), suggesting that ABCB5 blockade might potentiate growth inhibitory and pro-apoptotic effects of TMZ through revocation of G2/M cell cycle arrest.

The data presented herein demonstrate that treatment with TMZ triggered G2/M arrest in GBM cells by inducing phosphorylation and activation of ATM and CHEK1, inhibitory Ser216 phosphorylation of CDC25C, retention of inhibitory Tyr15 phosphorylation of CDK1, activation of the inhibitory kinases WEE1 and MYT1, and finally, accumulation of Cyclin B1, while ABCB5 blockade inhibited this arrest-inducing signaling. Treatment of GBM cells with TMZ in the presence of ABCB5 mAb removed the inhibitory phosphorylation of CDC25C and CDK1, thereby activating CDC25C and the Cyclin B1/CDK1 complex. This activation of CDC25C and CDK1 triggered by ABCB5 mAb-mediated inhibition of ATM and CHEK1 and by activation of PLK1 that inhibits the inhibitory kinases WEE1 and MYT1, represents a major positive regulator of G2/M transition. Abrogation of the G2/M checkpoint through inactivation of ATM and CHEK1 has been shown to sensitize GBM and other cancer cells to drug cytotoxicity. As a corollary to these reports, our data indicate that attenuation of TMZ-induced G2/M arrest by ABCB5 blockade sensitized GBM cells to drug-mediated death as was evident from increased apoptosis, and decreased proliferation of GBM cells upon combined treatment with TMZ and ABCB5 mAb.

Furthermore, there is significant inter-tumor heterogeneity in human GBM and GBM molecular subtypes are associated with diverse patient outcomes. Proneuronal or secondary GBM is characterized by mutations in IDH and TP53 with wild-type PTEN status (having features of neuronal differentiation and is associated with better outcomes) (Olar, A., and Aldape, K. D. (2014) Using the molecular classification of glioblastoma to inform personalized treatment. J Pathol 232, 165-177. Primary GBM possesses a mesenchymal molecular subtype with EGFR amplification and PTEN loss. It presents at an older age and typically has worse outcomes. It was shown herein that, while ABCB5 blockade exhibited significant anti-tumor activity in all cell lines in vitro, the in vivo inhibitory effect on tumor growth was most pronounced in TP53-mutant PTEN-wild type cancers.

The data disclosed herein define a novel role of ABCB5 in GBM and elucidate a molecular mechanism underlying ABCB5-mediated GBM tumor progression and chemoresistance. They support the role of ABCB5 targeting as a novel ‘two hit’ therapeutic strategy in combating GBM via conferring chemo-sensitivity as well as targeting those very subpopulations that drive tumor growth.

ABCB5 is an important marker for the isolation of drug resistant cancer cells. ABCB5(+), which functions as part of an intracellular signaling pathway involved in critical cellular regulation, is expressed on the cell surface and serves as a receptor for Phosphatidylinositol 4,5-bisphosphate (PtdIns(4,5)P2, also known simply as PIP2), and, to a lesser degree, PIP1 and PIP3. PIP2 is a minor phosphoinositol phospholipid component of cell membranes enriched at the plasma membrane, where it is a substrate for a number of important signaling proteins, regulating, for example, signaling through receptor tyrosine kinases (RTKs) through the PI3K pathway, or the IP3/DAG pathway of G-protein-coupled receptors. Inhibition of ABCB5-PIP2 pathway through inhibition of ABCB5, blocks PIP2 binding to ABCB5 and subsequently PIP2 phosphorylation to produce PIP3. Thus interruption of this pathway results in the inhibition of down-stream PI3K signaling of tyrosine kinase receptors (for example, VEGFR1, EGFR and AXL). ABCB5 binding of PIP2 can serve, among other functions, to increase its rate of phosphorylation to PIP3 and thus represents a stem cell-specific interaction to enhance the signaling roles of PIP2 in cells that do not express ABCB5. ABCB5-PIP2 binding can be inhibited by small molecule ABCB5 competitive ligands or substrates, or compositions comprising the same, which also inhibit downstream signaling of key ABCB5-dependent biological stem cell functions.

The invention can also be useful in the treatment of a subject having or at risk of having a disease, for example a subject having or at risk of having cancer. A subject shall mean a human or vertebrate mammal. Preferably the subject is a human. A subject at risk of developing a cancer is one who has a high probability of developing cancer. These subjects include, for instance, subjects having a genetic abnormality, the presence of which has been demonstrated to have a correlative relation to a higher likelihood of developing a cancer and subjects exposed to cancer causing agents such as tobacco, asbestos, or other chemical toxins, or a subject who has previously been treated for cancer and is in apparent remission. A subject at risk of having cancer also includes a subject having precancerous lesions. A precancerous lesion is an area of tissue that has altered properties and carries the risk of turning into skin cancer. Precancerous lesions may be caused by, for instance, UV radiation, genetics, exposure to carcinogens such as arsenic, tar or x-ray radiation.

A subject having a cancer is a subject that has detectable cancerous cells. The cancer may be a malignant or non-malignant cancer. Cancers or tumors include but are not limited to biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas. Preferably the cancer includes cancer cells that express ABCB5.

Optionally, prior to the treatment the presence of ABCB5 positive stem cells can be detected using the binding molecules described herein. The detection or diagnosis methods provided by the invention generally involve contacting one or more molecules of the invention with a sample in or from a subject. Preferably, the sample is first harvested from the subject, although in vivo detection methods are also envisioned. The sample may include any body tissue or fluid that is suspected of harboring the cancer stem cells. For example, the stem cells are commonly found in or around the tumor mass.

ABCB5 is a member of the ATP-binding cassette transporters sub-family B. It is a transmembrane protein encoded by the ABCB5 gene. ATP-binding cassette (ABC) transporters play a pivotal role in physiology and pathology. They are involved in the transport of structurally diverse molecules ranging from small ions, sugars, and peptides to more complex organic molecules.

The methods of the invention involve the disruption of ABCB5 in cancer cells utilizing synthetic compounds and naturally occurring substances that competitively inhibit PIP1, PIP2 or PIP3 binding to ABCB5 or disrupt ABCB5-dependent receptor tyrosine kinase and G Protein coupled receptor signal transduction, alone or in combination with anti-cancer drugs to treat the cancer.

Thus, in some aspects, compounds that competitively inhibit PIP1, PIP2 or PIP3 binding to ABCB5 and thus inhibit ABCB5-dependent signal transduction are useful according to the invention. In some embodiments these compounds include but are not limited to PtdIns-(1,2-dioctanoyl), a synthetic analog of natural phosphatidylinositol (PtdIns) containing C8:0 fatty acids at the sn-1 and sn-2 positions (CAS Registry Number 899827-36-2). These compounds are useful for treating ABCB5+ cancers.

In some aspects, ABCB5 inhibitors include, for example, PSC 833 (Valspodar), Zosuquidar, Tariquidar, and Laniquidar. In other embodiments, the ABCB5 inhibitors useful in the method of treating cancer is an ABCB1 agent for the treatment of heart disease/vessel disease. Non-limiting examples include any of the following compounds: Verapamil, Reserpine, Nifedipine, Digoxin, Quinidine, Nicardipine, Prazosin, Diltiazem, Amitriptyline, Losartan, Pravastatin, Acebutolol, Acetylsalicylic acid, Timolol, Nadolol, Debrisoquine, Ezetimibe, Tolvaptan, Pitavastatin, Canagliflozin, Clopidogrel, Ticagrelor, Apixaban, Cobimetinib, Selexipag, Ambrisentan, Metoprolol, Atenolol, Bromocriptine Amlodipine, Ivermectin, Clarithromycin, Ketoconazole, Ritonavir, Saquinavir, Nelfinavir, Indinavir, Rifampicin, Ciprofloxacin, Sparfloxacin, Levofloxacin, Grepafloxacin, Levomilnacipran, Simeprevir, idovudine, Atazanavir, Telaprevir, Fidaxomicin, Lamivudine, Sofosbuvir, voxilaprevir, Pibrentasvir, Glecaprevir, Letermovir, Omeprazole, Nizatidine, Domperidone, Lansoprazole, Ranitidine, Pantoprazole, and Dolutegravir.

Other miscellaneous ABCB5 inhibitors useful in the methods of the invention include but are not limited to: Ciclosporin, Cimetidine, Aldosterone, Tacrolimus, Phenobarbital, Dexamethasone, Carbamazepine, Colchicine, Loperamide, Imipramine, Hydrocortisone, Citalopram, Taurocholic Acid, Fexofenadine, Prednisone, Estrone, Diazepam, Digitoxin, Methylprednisolone, Quetiapine, Olanzapine, Clozapine, Prednisolone, Betamethasone, Alitretinoin, Vecuronium, Stanolone, Epinastine, Estriol, Sphingosine, Cerivastatin, Levetiracetam, Phenytoin, Lamotrigine, Sitagliptin, Ketazolam, Silodosin, Rivaroxaban, Dabigatran etexilate, Fesoterodine, Indacaterol, Clobazam, Linagliptin, Mirabegron, Bosutinib, Fluticasone furoate, Mycophenolate mofetil, Dapagliflozin, Umeclidinium, Edoxaban, Nintedanib, Ombitasvir, Elbasvir, Grazoprevir, Odanacatib, Baricitinib, Ubidecarenone, Ertugliflozin, Stanolone acetate, Estradiol acetate, Estradiol benzoate, Estradiol cypionate, Estradiol dienanthate, Estradiol valerate, Testosterone propionate, Asunaprevir, Somatostatin, Avatrombopag, Venlafaxine, Trimipramine, Tacrine, Eletriptan, Sumatriptan, Sirolimus, Paritaprevir, Dasabuvir, Erythromycin, Gramicidin D, Itraconazole, Tetracycline, Valinomycin, Topiramate, Terfenadine, Amprenavir, Celiprolol, Talinolol, Flupentixol, Trifluoperazine, Rhodamine 6G, Simvastatin, Valspodar, Cerliponase alfa, Curcumin, Ascorbic acid, Chlorpromazine, Phenothiazine, Atorvastatin, Bromperidol, Morphine, Pentazocine, Propranolol, Neostigmine, Moxidectin, Mefloquine, Fluticasone, Fluticasone propionate, Elagolix, Chloroquine, Paliperidone, Lusutrombopag, Posaconazole, Dipyridamole, Quinine, Indometacin, Acetaminophen, Haloperidol, Naloxone, Mannitol, Betrixaban, Clomifene, Omadacycline, Grapiprant, Larotrectinib, Revefenacin, Tenofovir disoproxil, Tenofovir alafenamide, Tenofovir, Ledipasvir, Sildenafil, Vardenafil, Cabergoline, Prucalopride, Risperidone, Tramadol, Azithromycin, Fluconazole, Ranolazine, Cetirizine, Tegaserod, and Doxepin.

In some embodiments, the ABCB5 inhibitor is a PIP2 antagonist, which may be a small molecule or a lipid analog.

In some embodiments, the ABCB5 inhibitor is an anti-ABCB5 antibody or fragment. In some embodiments, the ABCB5 antibody is selected for example from a list comprising, monoclonal antibodies, polyclonal antibodies, human antibodies, chimeric antibodies, humanized antibodies, single-chain antibodies, F(ab′)2, Fab, Fd, Fv or single-chain Fv fragments.

In some embodiments, the ABCB5 antibody is a human anti-ABCB5 antibody or ABCB5-binding fragment that binds to an extracellular loop of a three dimensional configuration of ABCB5. In some embodiments the human anti-ABCB5 antibody is subjected to an affinity maturation to recognize and bind specifically to the extracellular loop of a non-linear form of ABCB5. The human anti-ABCB5 antibody or ABCB5-binding fragment described herein has a sequence that corresponds to an antibody preparable by a method comprising affinity maturation to bind specifically to the extracellular loop of a non-linear form of the ABCB5.

In some embodiments the antibody is an ABCB5 antibody or ABCB5-binding fragment having specificity for the cyclical form or the linear form of an extracellular polypeptide of the ABCB5. In some embodiments, the composition comprises an ABCB5 antibody or ABCB5-binding fragment that alters the conformation of ABCB5 PIP2 binding site.

In other embodiments the antibody is a bispecific antibody. that is specific for ABCB5 and RTKs. Bispecific antibodies may resemble single antibodies (or antibody fragments) but have two different antigen binding sites. Bispecific antibodies may have binding specificities for at least two different epitopes. Bispecific antibodies and fragments can also be in form of heteroantibodies. Heteroantibodies are two or more antibodies, or antibody binding fragments (e.g., Fab) linked together, each antibody or fragment having a different specificity.

The ABCB5-RTK bispecific antibody modulates a signalling pathway. In some embodiments, modulation of a signaling pathway using the bispecific antibody includes a change in the signaling pathway activity by at least 10%, 20%, 30%, 40%, 50%, 60%, 70%, 80%, 90%, 99% or 99.9% as compared to no antibody or using a monospecific antibody. In some embodiments, upregulation of a signaling pathway includes turning on or initiating the pathway that was off or substantially not active. In some embodiments, downregulation of a target signaling pathway can include turning off or substantially blocking the pathway that was on or substantially active. The modulation of the one or more signaling pathway may lead to certain changes in target cell(s)'s behavior, such as reducing cell proliferation, cell growth, cell differentiation, cell survival, or cell secretion.

Antibody conjugates are also provided. The conjugates include any antibody of the present disclosure and an agent. The agent may be selected from a therapeutic agent, an imaging agent, a labeling agent, or an agent useful for therapeutic and/or labeling purposes.

The strength or affinity of binding interactions between an antibody (or bispecific version or fragment thereof) and the specific antigen (or epitope) can be expressed in terms of the dissociation constant (K_{D}) of the interaction, wherein a smaller K_{D} represents a greater affinity.

The term “specific binding” or “antigen-specific antibody” refers to the ability of an antibody to preferentially bind to a particular antigen that is present in a mixture of different antigens. In certain embodiments, a specific binding interaction will discriminate between desirable and undesirable antigens (or “target” and “non-target” antigens) in a sample, in some embodiments more than about 10 to 100-fold or more (e.g., more than about 1000- or 10,000-fold). In certain embodiments, the affinity between an antibody and antigen when they are specifically bound in an antibody-antigen complex is characterized by a K_{D} (dissociation constant) of less than 10^{-6M}, less than 10^{-7 M}, less than 10−^{8 M}, less than 10^{-9 M}, less than 10^{-10 M}, less than 10^{-11M}, or less than about 10^{-12M} or less.

In some embodiments the ABCB5 inhibitor is an inhibitory nucleic acid. Various strategies for gene knockdown by inhibitory nucleic acids are known in the art. For example, gene knockdown strategies may be used that make use of RNA interference (RNAi) and/or microRNA (miRNA) pathways including small interfering RNA (siRNA), short hairpin RNA (shRNA), double-stranded RNA (dsRNA), miRNAs, and other small interfering nucleic acid-based molecules known in the art. In one embodiment, vector-based RNAi modalities (e.g., shRNA or shRNA-mir expression constructs) are used to reduce expression of a gene in a cell. In some embodiments, therapeutic compositions of the invention comprise an isolated plasmid vector (e.g., any isolated plasmid vector known in the art or disclosed herein) that expresses a small interfering nucleic acid such as an shRNA. The isolated plasmid may comprise a tumor-specific, e.g., GBM-specific, promoter operably linked to a gene encoding the small interfering nucleic acid, e.g., an shRNA. In some cases, the isolated plasmid vector is packaged in a virus capable of infecting the individual. Exemplary viruses include adenovirus, retrovirus, lentivirus, adeno-associated virus, and others that are known in the art and disclosed herein.

The compounds, antibodies, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art.

The pharmaceutical compositions to be used in the present methods can comprise pharmaceutically acceptable carriers, excipients, or stabilizers in the form of lyophilized formulations or aqueous solutions. Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some examples, the pharmaceutical composition described herein comprises liposomes containing the compounds or antibodies. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The compounds or antibodies, or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions.

In other examples, the pharmaceutical composition described herein can be formulated in sustained-release format. Suitable examples of sustained-release preparations include semipermeable matrices of solid hydrophobic polymers containing the compounds or antibody, which matrices are in the form of shaped articles, e.g. films, or microcapsules.

The pharmaceutical compositions described herein can be in unit dosage forms such as tablets, pills, capsules, powders, granules, solutions or suspensions, or suppositories, for oral, parenteral or rectal administration, or administration by inhalation or insufflation. For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%.

The emulsion compositions can be those prepared by mixing a compound or an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

Any of the compounds or antibodies, as well as the encoding nucleic acids or nucleic acid sets, vectors comprising such, or host cells comprising the vectors, described herein are useful for treating cancer. To practice the method disclosed herein, an effective amount of the pharmaceutical composition described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the compounds or antibodies as described herein can be aerosolized using a fluorocarbon formulation and a metered dose inhaler, or inhaled as a lyophilized and milled powder.

Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for a compound or an antibody as described herein may be determined empirically in individuals who have been given one or more administration(s) of the compound or antibody. Individuals are given incremental dosages of the compound. To assess efficacy of the compound, an indicator of the disease/disorder can be followed.

Generally, for administration of any of the compounds or antibodies described herein, an initial candidate dosage can be about 2 mg/kg. For the purpose of the present disclosure, a typical daily, weekly, every two weeks, or every three weeks dosage might range from about any of 0.1 μg/kg to 3 μg/kg to 30 μg/kg to 100 μg/kg to 300 μg/kg to 0.6 mg/kg, 1 mg/kg, 3 mg/kg, to 10 mg/kg, to 30 mg/kg to 100 mg/kg or more, depending on the factors mentioned above. For repeated administrations over several days, weeks, months, or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 3 mg/kg every 3 weeks, followed by a maintenance dose of about 1 mg/kg of the compound or antibody once in 6 weeks, or followed by a maintenance dose of about 1 mg/kg every 3 weeks. However, other dosage regimens may be useful, depending on the pattern of pharmacokinetic decay that the practitioner wishes to achieve. For example, dosing of 1 mg/kg once in every 3 weeks in combination treatment with at least one additional immune therapy agent is contemplated. In some embodiments, dosing ranging from about 3 μg/mg to about 3 mg/kg (such as about 3 μg/mg, about 10 μg/mg, about 30 μg/mg, about 100 μg/mg, about 300 μg/mg, about 1 mg/kg, and about 3 mg/kg) may be used. In some embodiments, dosing frequency is once every week, every 2 weeks, every 3 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the compound or antibody used) can vary over time.

In some embodiments, for an adult patient of normal weight, doses ranging from about 0.1 to 5.0 mg/kg may be administered. In some examples, the dosage described herein can be 10 mg/kg. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of a compound or antibody as described herein will depend on the specific compound or antibody, antibodies, and/or non-antibody peptide (or compositions thereof) employed, the type and severity of the disease/disorder, whether the compound or antibody is administered for preventive or therapeutic purposes, previous therapy, the patient's clinical history and response to the antagonist, and the discretion of the attending physician. Typically the clinician will administer a compound or an antibody, until a dosage is reached that achieves the desired result. In some embodiments, the desired result is a reduction of the size of the tumor, increased progression-free survival period and/or overall survival. Methods of determining whether a dosage resulted in the desired result would be evident to one of skill in the art. Administration of one or more compounds or antibodies can be continuous or intermittent, depending, for example, upon the recipient's physiological condition, whether the purpose of the administration is therapeutic or prophylactic, and other factors known to skilled practitioners. The administration of a compound or an antibody may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder. Alleviating a target disease/disorder includes delaying the development or progression of the disease, or reducing disease severity. Treatment decreases the likelihood that the subject will develop the disease as well as a treatment after the subject has developed the disease in order to fight the disease, prevent the disease from becoming worse, or slow the progression of the disease compared to in the absence of the therapy.

Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

In some embodiments, the compounds or antibodies described herein are administered to a subject in need of the treatment at an amount sufficient to inhibit the activity of ABCB5 or other products in the ABCB5-PIP2 pathway by at least 20% (e.g., 30%, 40%, 50%, 60%, 70%, 80%, 90% or greater) in vivo. In other embodiments, the compound or antibody is administered in an amount effective in reducing the activity level of ABCB5 or other products in the ABCB5-PIP2 pathway by at least 20% (e.g., 30%, 40%, 50%, 60%, 70%, 80%, 90% or greater).

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered parenterally, topically, orally, by inhalation spray, rectally, nasally, buccally, vaginally or via an implanted reservoir. The term “parenteral” as used herein includes subcutaneous, intracutaneous, intravenous, intraperitoneal, intratumor, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble compounds or antibodies can be administered by the drip method, whereby a pharmaceutical formulation containing the compounds or antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the compounds or antibody, can be dissolved and administered in a pharmaceutical excipient such as Water-for-Injection, 0.9% saline, or 5% glucose solution.

In some embodiments, more than one compound or antibody, or a combination of a compound or an antibody and another suitable therapeutic agent, may be administered to a subject in need of the treatment. The compounds or antibody can also be used in conjunction with other agents that serve to enhance and/or complement the effectiveness of the agents.

Treatment efficacy for a target disease/disorder can be assessed by methods well-known in the art.

The treatment methods involving such as described in the present disclosure may be utilized in conjunction with other types of therapy for the target disease or disorder disclosed herein. Examples include chemotherapy, immune therapy (e.g. therapies involving therapeutic antibodies, antibodies, CAR T cells, or cancer vaccines), surgery, radiation, gene therapy, and so forth, or anti-infection therapy. Such therapies can be administered simultaneously or sequentially (in any order) with the treatment according to the present disclosure. In some instance, the target disease is cancer (e.g., those disclosed herein) and the conjunction therapy involves an immune checkpoint (e.g., inhibitory checkpoint) antagonist. Examples include PD-1/PD-L1 antagonists (e.g., nivolumab, pembrolizumab, avelumab, durvalumab and atezolizumab), LAG3 antagonists, TIM-3 antagonists, VISTA antagonists, TIGIT antagonists, CSF1R antagonists, CD112R (PVRIG) antagonists, PVR (CD155) antagonists, PD-L2 antagonists, A2AR antagonists, B7-H3 antagonists, B7-H4 antagonists or BTLA antagonists. Additional examples include activators that enhance the activity of stimulatory checkpoint such as CD122 (IL2) agonist, 4-1BB, ICOS ligand, GITR, and OX40.

When co-administered with an additional therapeutic agent, suitable therapeutically effective dosages for each agent may be lowered due to the additive action or synergy.

The efficacy of the methods described herein may be assessed by any method known in the art and would be evident to a skilled medical professional. For example, the efficacy of the antibody-based immunotherapy may be assessed by survival of the subject or cancer burden in the subject or tissue or sample thereof. In some embodiments, the methods are assessed based on the safety or toxicity of the therapy in the subject, for example by the overall health of the subject and/or the presence of adverse events or severe adverse events.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Unless otherwise defined herein, scientific and technical terms used in connection with the present disclosure shall have the meanings that are commonly understood by those of ordinary skill in the art. Further, unless required by context, singular terms shall include pluralities and plural terms shall include the singular. The methods and techniques of the present disclosure are generally performed according to conventional methods well-known in the art. Generally, nomenclature used in connection with, and techniques of biochemistry, enzymology, molecular and cellular biology, microbiology, genetics and protein and nucleic acid chemistry and hybridization described herein are those well-known and commonly used in the art. The methods and techniques of the present disclosure are generally performed according to conventional methods known in the art and as described in various general and more specific references that are cited and discussed throughout the present specification unless otherwise indicated.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Materials and Methods

GlioVis. Glioblastoma and lower grade glioma adult brain tumor copy number alteration and RNA-seq datasets from TCGA were queried for ABCB5 and PROM1 (CD133), while glioblastoma survival and patient outcomes based on subtype were queried for ABCB5 expression from RNA-seq datasets.

Cell culture. Authenticated human GBM cell lines U-87 MG, LN-18 and LN-229 were obtained from American Type Culture Collection (ATCC, Manassas, Va.). LN-18 and LN-229 were cultured in DMEM, and U-87 MG in EMEM (ATCC, Manassas, Va.) supplemented with 10% (v/v) FBS (Invitrogen GIBCO, Waltham, Mass.) and 1% (v/v) penicillin/streptomycin (Lonza Bio-Whittaker, Walkersville, Md.). For antibody-mediated inhibition of ABCB5, GBM cells were incubated with designated doses of anti-ABCB5 monoclonal antibody (mAb) (clone 3C2-1D12) or MOPC31C isotype control mAb (Sigma-Aldrich, St. Louis, Mo.) for 72 hours. All experiments were performed with cell lines that tested negative for mycoplasma (Lonza, Portsmouth, N.H.).

RNA extraction and RT PCR. RNA was prepared from U-87 MG, LN-18 and LN-229 GBM or G3361 melanoma cells using a RNeasy Plus isolation kit (Qiagen, Germantown, Md.) and reverse-transcribed using an Advantage RT-for-PCR Kit (Clontech, Mountain View, Calif.) according to the manufacturers' instructions. cDNA was then subjected to PCR amplification of the full ABCB5 open reading frame (ORF; transcript variant 2, mRNA NCBI Reference Sequence: NM_178559.5) as previously described for human skin cells. For sequencing reactions, the PCR product was then used as a template for nested PCR. All PCRs used Q5 high-fidelity polymerase (NEB, Ipswich, Mass.). The full ORF sequences of ABCB5 (transcript variant 2, mRNA NCBI Reference Sequence: NM_178559.5) expressed by the human LN-229, LN-18, and U-87 MG glioblastoma cell lines were submitted to the GenBank database under the following accession numbers: MK803369, MK803368, MK803366, MK803367.

Flow cytometric analyses of ABCB5 and CD133 expression. U-87 MG, LN-18 and LN-229 GBM cells were harvested with Versene (Invitrogen GIBCO, Waltham, Mass.) and stained with FITC-conjugated mouse monoclonal anti-ABCB5 antibody (clone 3C2-1D12) or CFS-conjugated mouse IgG1 isotype control (R&D Systems, Minneapolis, Minn.) and APC-conjugated CD133/2 (293C3) (Miltenyi Biotech, Cambridge, Mass.) or APC-conjugated mouse isotype control (R&D Systems, Minneapolis, Minn.).

Generation of stable ABCB5 knockdown glioblastoma cell variants. Generation of stable LN-229 and U-87 MG ABCB5 knockdown (KD) or their respective shControl cell variants was accomplished followed by puromycin selection (2 μg/mL). Reduction of ABCB5 protein expression in ABCB5 KD cell lines was confirmed by immunoprecipitation (IP)-Western blot. Briefly, 5 mg each of sonicated and precleared total cell lysates were prepared in RIPA buffer (Boston BioProducts, Ashland, Mass.) plus Protease Inhibitor (Sigma-Aldrich, St. Louis, Mo.) before incubation with 2 μg of anti-ABCB5 rabbit pAb (Abgent/Abcepta, San Diego, Calif.) with protein A/G agarose for 3 hours at 4° C. After washing 3 times, SDS-Page and Western blotting was performed, using the same antibody.

Cell proliferation assay. Cell proliferation was measured by MTT Cell Proliferation Assay Kit (Trevigen, Gaithersburg, Md.) following the manufacturer's protocol. Briefly, 1×10⁴ cells were seeded in 100 μl culture medium per well of 96-well plates. Following treatment with anti-ABCB5 mAb or isotype control mAb in absence or presence of TMZ, 10 μl of MTT reagent was added to each well. Once purple crystals of formazan were visible, 100 μl of Detergent Reagent was added, and the cells were incubated in the dark for 2-4 hours until the crystals became soluble. Absorbance was measured at 570 nm and corrected against blank wells, which consisted of culture medium alone and were processed in the same way as above.

In vivo tumor xenograft study. 6-week-old female NOD/SCID IL2rγ^{-/-} (NSG) mice purchased from the Jackson Laboratory (Bar Harbor, Me.) were maintained in accordance with the institutional guidelines of Boston Children's Hospital and Harvard Medical School and experiments were carried out according to approved experimental protocols. Human xenografts were established by subcutaneous injection of human GBM LN-229 and U-87 MG cells into the right flank of recipient NSG mice (5×10⁶ cells/inoculum). For determining the effect of ABCB5 blockade on tumor growth, the mice were injected intraperitoneally with 1 mg anti-ABCB5 mAb or 1 mg isotype control mAb three times per week starting one week prior to tumor inoculation. To determine the effect of ABCB5 blockade on TMZ (Sigma-Aldrich, St. Louis, Mo.) sensitization of the xenograft tumors, LN-229 GBM xenografts were established in NSG mice as described above. Once the tumors reached the volume of 100 mm³, the mice were randomized into 4 groups (n=6 per group) that received either 1 mg anti-ABCB5 mAb or 1 mg isotype control mAb in the presence of either 0.1 mg TMZ or its vehicle. Intraperitoneal injection of anti-ABCB5 mAb or the isotype control mAb was started a week before the initiation of daily intraperitoneal injections of TMZ. TMZ was freshly dissolved in 10% DMSO and diluted in saline before every injection. Tumor volumes were measured twice every week according to the established formula tumor volume (mm³)=π/6×0.5×length×(width)².

Immunohistochemistry. Immunohistochemical staining for Ki-67 and cleaved Caspase-3 on deparaffinized 5-μm sections of GBM tumor xenografts was done as described previously (Wilson, B. J., et al., (2011) ABCB5 identifies a therapy-refractory tumor cell population in colorectal cancer patients. Cancer Res 71, 5307-5316), using anti-Ki67 (Vector Laboratories, Burlingame, Calif.) and anti-cleaved caspase-3 (Cell Signaling Technology, Danvers, Mass.) antibodies. For quantitative analyses of Ki-67 and cleaved caspase-3 expression in the xenograft tumors, slides were scanned via Aperio Digital Pathology Service provided by the Research Pathology Cores, Dana-Farber/Harvard Cancer Center, Boston, Mass. Nuclear staining algorithms were utilized for enumeration of the positive cells.

Apoptosis assay. GBM cells were treated with anti-ABCB5 mAb or isotype control mAb in the absence or presence of TMZ for 72 h. Induction of apoptosis by antibody-mediated ABCB5 blockade was detected by flow cytometry using APC Annexin V and PI (BD Biosciences, Billerica, Mass.) staining as per the manufacturer's protocol.

Microarray analyses. Microarray analyses were performed by the Microarray Core Facility at the Dana-Farber Cancer Institute using HTA 2.0 human arrays. FACS-sorted ABCB5-positive and ABCB5-negative cells isolated from U-87 MG, LN-18, and LN-229 GBM cell lines were compared. Data were preprocessed using the R Bioconductor oligo package with the RMA normalization method. Differentially expressed genes (DEGs) were identified using the R Bioconductor limma package with predefined criteria, followed by input of these genes into Ingenuity Pathway Analysis.

Cell cycle analysis. Cell cycle distribution was determined by flow cytometry using PI/RNase staining buffer (BD Biosciences, Billerica, Mass.) following the manufacturer's protocol. Briefly, harvested cells were washed in PBS and fixed in ice cold 70% ethanol overnight. The fixed cells were washed in PBS and resuspended in PI/RNase staining buffer. Following incubation at 37° C. for 30 min, the fractions of cells in G0/G1, S, and G2/M phase were analyzed by flow cytometry at an excitation wavelength of 488 nm and an emission wavelength of 630 nm.

Western blot. Protein lysates were prepared from cultured GBM cells or excised human GBM xenograft tumors in RIPA buffer (Cell Signaling Technology, Danvers, Mass.) supplemented with protease inhibitor cocktail (Sigma-Aldrich, St. Louis, Mo.). Normalized protein lysates were run on SDS-polyacrylamide gels and transferred to a polyvinylidene difluoride membrane (GE Healthcare Life Sciences, Pittsburgh, Pa.). The membranes were incubated with either rabbit anti-β-Actin (D6A8), mouse anti-β-Actin (8H10D10) anti-phospho-ATM (Ser1981) (D6H9), anti-ATM (D2E2), anti-phospho-CHEK1 (Ser345) (133D3), anti-CHEK1 (2G1D5), anti-phospho-CDC25C (Ser216) (63F9), anti-CDC25C (5H9), anti-phospho-PLK1 (Thr210) (D5H7), anti-PLK1 (208G4), anti-Cyclin B1 (D5C10), anti-phospho-CDC2 (Tyr15) (10A11), anti-CDC2, anti-phospho-WEE1 (Ser642) (D47G5), anti-WEE1 (D10D2) or anti-MYT1 (Cell Signaling Technology, Danvers, Mass.), and subsequently incubated with peroxidase-linked secondary antibody. The reactive bands were detected using chemiluminescent substrate (Thermo Scientific, Waltham, Mass.).

Capillary Western blot analyses. Capillary Western analyses were performed on a Western blot system (ProteinSimple) according to the manufacturer's instructions. In brief, protein lysates prepared from cultured GBM cells in RIPA buffer and diluted to 2 μg/μL in sample buffer. The diluted samples were combined with fluorescent master mix and heated for 5 min at 95° C. The prepared samples, blocking reagent, primary antibodies (1:20 dilution for CDC25C, PLK1, Cyclin B1, CDC2, WEE1 and MYT1, 1:10 dilution for CHEK1, 1:5 dilution for phospho-CHEK1, phospho-CDC25C, phospho-PLK1, phospho-CDC2 and phospho-WEE1, and 1:80 dilution for mouse and rabbit-β-Actin), secondary antibodies, and chemiluminescent substrate were pipetted into designated wells in the assay plate. The electrophoresis and immunodetection steps were carried out in the fully automated capillary system. Data were analyzed using Compass software (ProteinSimple).

Statistics. The data are expressed as mean±SEM of three or more independent experiments. A Kruskal-Wallis test with Dunn's multiple comparisons test with a single pooled variance was used to determine statistical difference in the TCGA RNA-seq data, with P<0.05 considered significant. To determine the cutoff between high and low ABCB5 expression in the GBM RNA-seq data and for the different GBM subtypes, the R package maxstat was used to identify the cutpoint based on the maximally selected rank statistic. Statistical difference in expression level of markers, percentage of apoptotic cells, DNA content of cells, and tumor weight between different groups was determined by unpaired and paired t-test, with P<0.05 considered significant. Statistical difference in cell growth kinetics or tumor growth kinetics between different groups was determined using two-way ANOVA and Sidak's multiple comparisons test, with P<0.05 considered significant.

Data Availability. The full ORF sequences of ABCB5 (transcript variant 2, mRNA NCBI Reference Sequence: NM_178559.5) expressed by the human LN-229, LN-18, and U-87 MG glioblastoma cell lines were submitted to the GenBank database under the following accession numbers: MK803369, MK803368, MK803366, MK803367. The microarray analyses were deposited to the Gene Expression Omnibus under GSE127895 accession number.

### Example 1: ABCB5 is Expressed in GBM Primary Tumors and Cell Lines

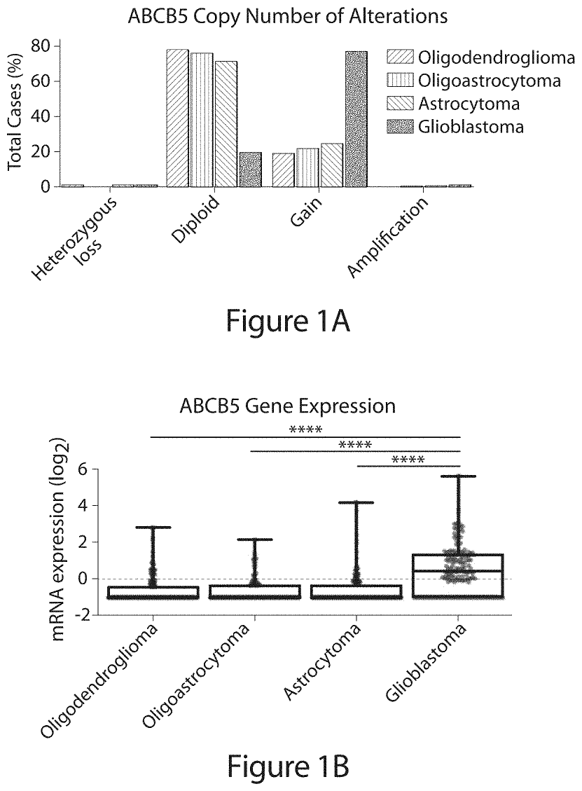

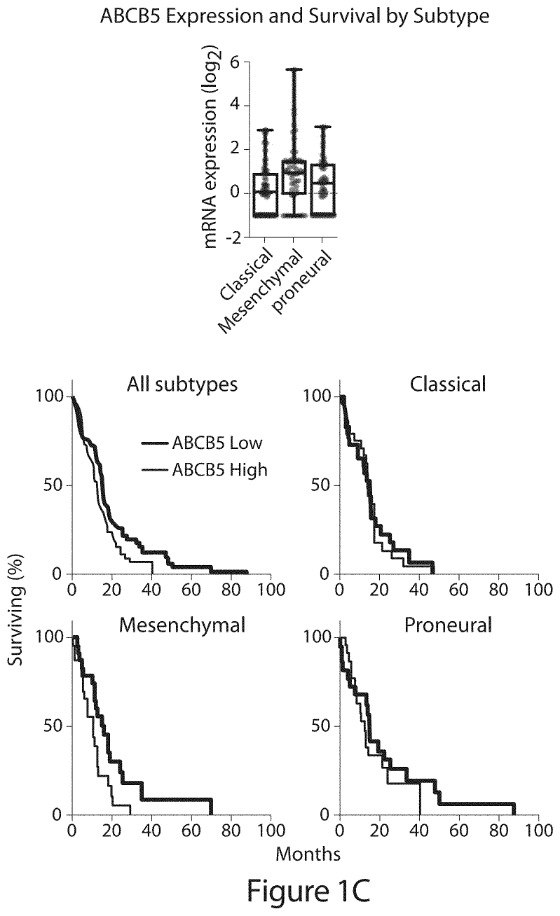

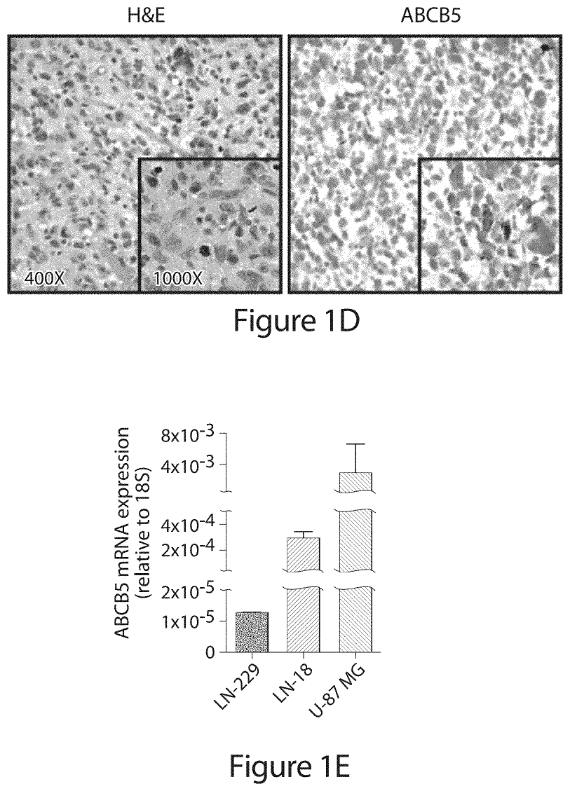

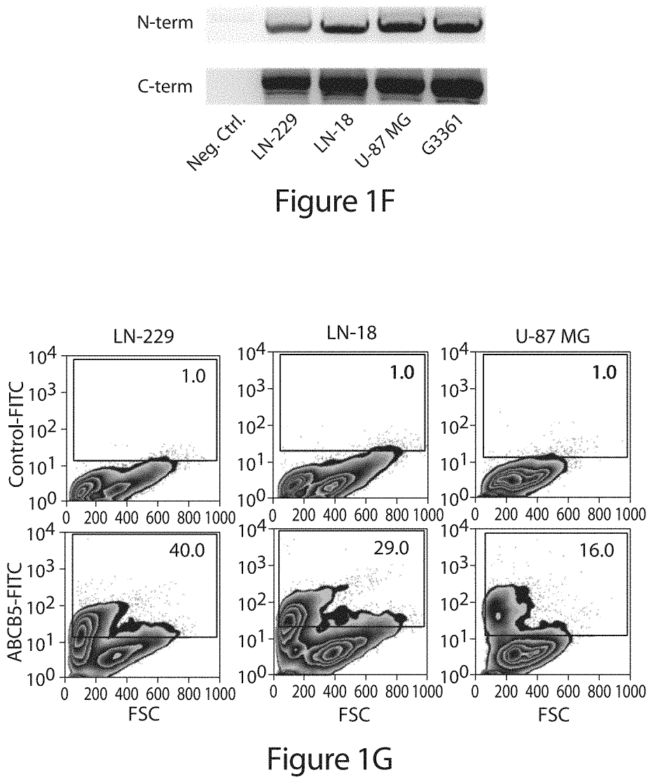

ABCB5 copy number alteration was examined. Expression in GBM and lower grade glioma (LGG) sequencing data from The Cancer Genome Atlas (TCGA) was determined. An enhanced increase in copy number gains was seen in GBM (77.4%) compared to oligodendroglioma (19.6%), oligoastrocytoma (22.5%), and astrocytoma (25.3%) (FIG. 1A). There was a statistically significant difference between means of ABCB5 mRNA expression in various brain cancers (H=138.1, P<0.0001). ABCB5 mRNA expression was significantly higher in GBM (0.51±0.11 log₂(counts), mean±SEM) compared to oligodendroglioma (−0.66±0.05 log₂(counts), P<0.0001), oligoastrocytoma (−0.62±0.06 log₂(counts), P<0.0001), and astrocytoma (−0.64±0.06 log₂(counts), P<0.0001) (FIG. 1B). There was no statistically significant difference in ABCB5 mRNA expression between oligodendroglioma and oligoastrocytoma (P>0.99), oligoastrocytoma and astrocytoma (P>0.99), or oligodendroglioma and astrocytoma (P>0.99). ABCB5 was detected in the GBM TCGA mRNA expression dataset in all three GBM subtypes (classical: 0.16±0.15 log₂(counts), mesenchymal: 0.90±0.20 log₂(counts), proneural: 0.48±0.18 log₂(counts), mean±SEM) (FIG. 1C, left).). We also examined the correlation of ABCB5 mRNA expression with overall survival (OS) for all GBM subtypes (FIG. 1C, right). There was a significant trend for median OS to be lower (12.6 vs. 14.9 months) for high compared to low ABCB5 expression (optimal cutoff high/low=log₂(0.06), P=0.053) when all GBM subtypes were considered together. For the mesenchymal subtype, median survival was significantly lower (10.4 vs.15.9 months) for high versus low ABCB5 expression (optimal cutoff=log₂(0.92), P=0.031). For the classical and the proneural subtypes, median survival was nominally lower, with 14.0 vs. 14.9 months (high vs. low ABCB5 expression, optimal cutoff=log₂(1.55), P=0.953) and 12.5 vs. 14.9 months (high vs. low ABCB5 expression, optimal cutoff=log₂(0.03), P=0.453), respectively. These results further underscored the clinical significance of ABCB5 expression in GBM, most notably for the mesenchymal subtype. Immunohistochemical analysis confirmed ABCB5 expression in patient-derived primary GBM tumor biopsy material at the protein level (FIG. 1D).

It has been recognized that GBM exhibits substantial inter-tumor heterogeneity and can be distinguished as primary GBM, i.e., presenting as fully developed high-grade gliomas without evidence of a precursor lesion, or secondary GBM, which evolves from less malignant astrocytomas. Furthermore, TCGA analyses identified the existence of distinct GBM molecular subtypes based on their genomic alterations and characteristic molecular signatures. Among them, a proneural subtype with mutations in IDH and TP53 corresponds to secondary GBM and is associated with a better outcome. A mesenchymal subtype that carries homozygous PTEN loss corresponds to primary GBM and is associated with a worse outcome. Here, we examined ABCB5 expression and function in GBM cell lines representative of such molecular subtypes, i.e. the LN-229 and LN-18 cell lines that carry mutations in TP53 and are PTEN wild-type, and the U-87 MG cell line that is characterized by PTEN loss and wild-type TP53. We found ABCB5 to be expressed in all three human GBM cell lines (LN-229, LN-18, and U-87 MG) by qRT-PCR (FIG. 1E), nested RT-PCR (FIG. 1F) and by flow cytometric analysis (FIG. 1G), which showed cell surface expression of ABCB5 on 40.6% (40.6±0.8%, mean±SEM) of LN-229, 31.8% (31.8±2.1%, mean±SEM) of LN-18 and 16.5% (16.5±0.9%, mean±SEM) of U-87 MG GBM cells.

### Example 2: ABCB5 is Expressed by CD133-Positive GBM Stem Cells and Antibody-Mediated ABCB5 Blockade Reduces the Frequency of CD133-Positive Stem Cells

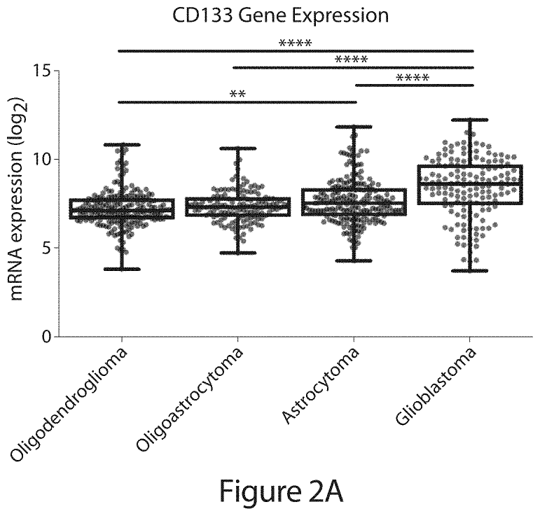

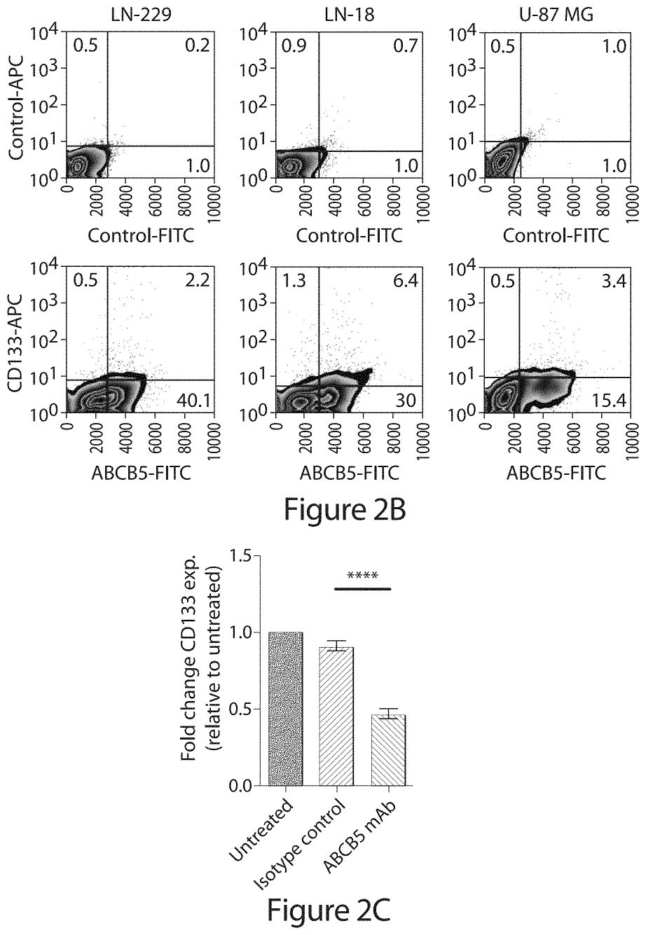

Based on the finding of ABCB5 expression on a subpopulation of GBM cells (FIG. 1G), it was hypothesized that ABCB5 would confer therapeutic resistance on GBM CSCs, which are marked by CD133. To test this further, CD133 mRNA expression in GBM and LGG RNA-seq data from TCGA was first examined. Similar to ABCB5, there was a statistically significant difference in CD133 mRNA expression between means (H=71.46, P<0.0001). CD133 mRNA expression was significantly higher in GBM (8.39±0.14 log₂(counts), mean±SEM) compared to oligodendroglioma (7.21±0.08 log₂(counts), P<0.0001), oligoastrocytoma (7.34±0.08 log₂(counts), P<0.0001), and astrocytoma (7.64±0.09 log₂(counts), P<0.0001) (FIG. 2A). There was no statistically significant difference in expression between oligodendroglioma and oligoastrocytoma (P>0.99) or oligoastrocytoma and astrocytoma (P=0.40). Additionally, a statistically significant difference was observed between oligodendroglioma and astrocytoma (P=0.004). CD133 expression positively correlated with ABCB5 in GBM and LGG TCGA RNA-seq data (rₛ(667)=0.26, P<0.0001). Moreover, dual-color flow cytometry confirmed co-expression of ABCB5 and CD133 on 2.2% of LN-229, 6.4% of LN-18 and 3.4% of U-87 MG human GBM cells (FIG. 2B). Furthermore, 100% of CD133-positive LN-229 and U-87 MG cells and 93% of CD133-positive LN-18 cells expressed ABCB5. Considering the evidence for the tumorigenic potential of CD133-positive GBM stem cells, expression of ABCB5 on CD133-positive CSCs indicates the possibility of targeting GBM growth by functionally inhibiting ABCB5.

Alongside the discovery of a novel anti-apoptotic function of ABCB5 in normal stem cells and human colorectal cancer, it was hypothesized that ABCB5 might also be required for GBM stem cell maintenance and tumor aggressiveness. When LN-229, LN-18, and U-87 MG cells were incubated with 100 μg/ml anti-ABCB5 monoclonal antibody (mAb) (3C2-1D12) or isotype-matched control mAb for 72 hours and expression of CD133 (APC, FL4 fluorescence) was determined by flow cytometry, we found that in vitro treatment of the GBM cell lines LN-229 and LN-18 with anti-ABCB5 mAb reduced the frequency of CD133-positive CSC subpopulations by more than 2-fold compared to treatment with isotype control (ABCB5 mAb vs. isotype control: 0.35±0.03% vs. 0.83±0.03%, P<0.0001, mean±SEM) (FIG. 2C), while no significant difference in CD133-positive cell frequency was observed in U-87 MG cells after treatment with anti-ABCB5 mAb (ABCB5 mAb vs. isotype control: 1.24±0.02% vs. 1.02±0.13%, P=0.1664, mean±SEM). These differential responses might be explained by the genetic heterogeneity of the GBM cell lines under study. For example, the LN229 and LN18 cell lines have increased mutation burden in DNA repair genes such as TP53 and wild-type PTEN status compared to U-87 MG, which is TP53 wild-type and PTEN mutant. Moreover, these results suggest that, in U-87 MG, CD133-positive GBM stem cells might utilize additional ABCB5-independent pathways for their survival and could show attenuated responses to ABCB5 blockade in vivo.

### Example 3: Antibody-Mediated ABCB5 Blockade Inhibits Proliferation, and Promotes Apoptosis in Human GBM

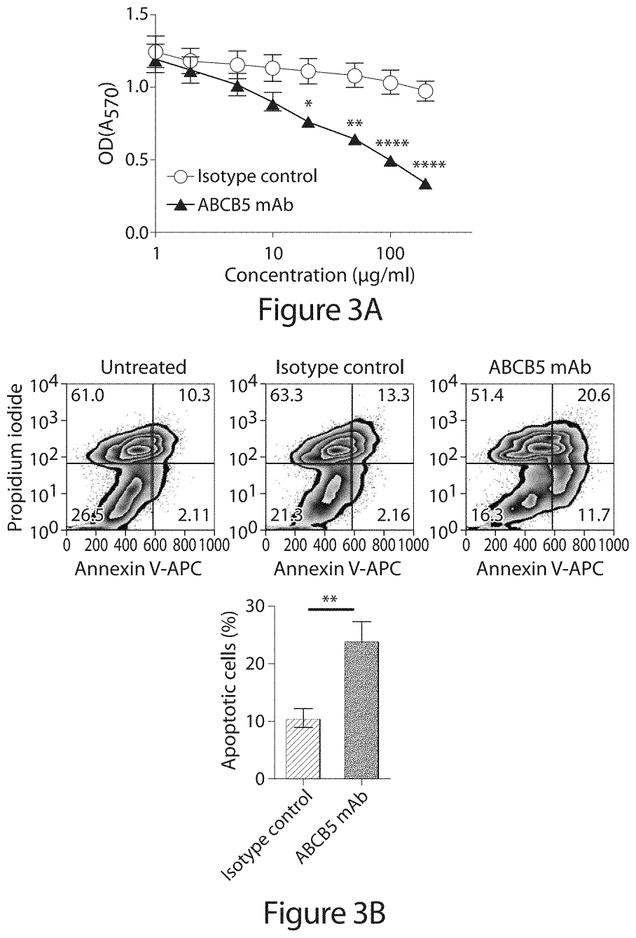

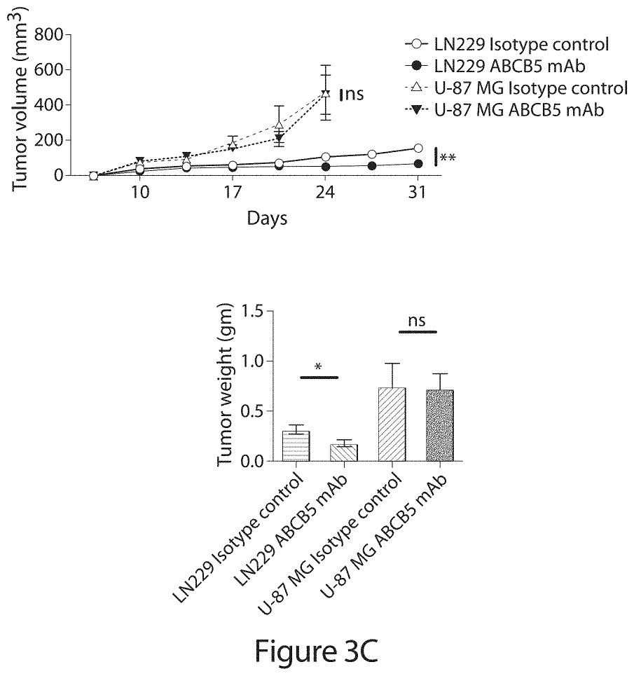

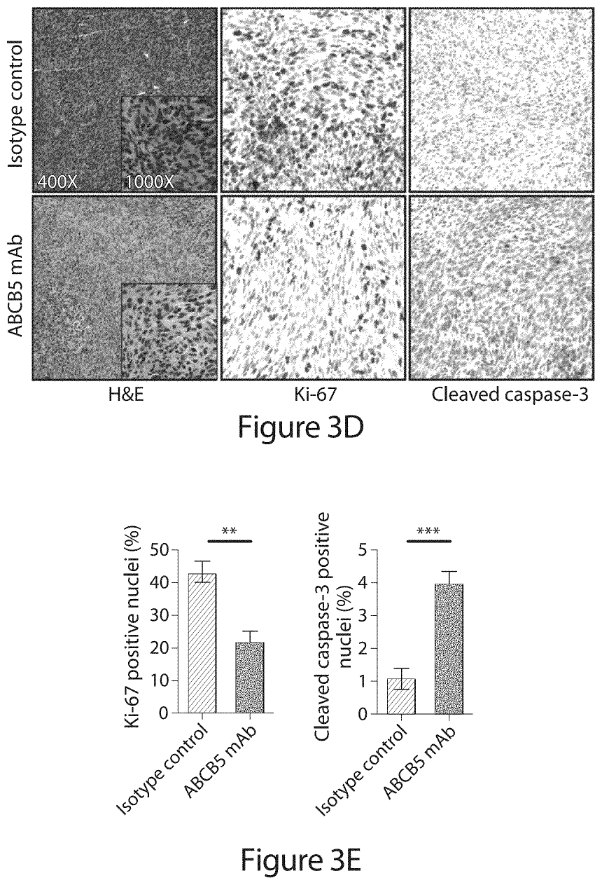

Considering the significance of CD133-positive CSCs in the growth of GBM, the effect of the reduction in CSC frequency on GBM cell proliferation was examined. We incubated LN-229, LN-18, and U-87 MG cells with 0-200 μg/ml ABCB5 mAb or isotype control mAb for 72 hours and analyzed cell proliferation by 3-(4,5-Dimethylthiazol-2-yl)-2,5-diphenyltetrazolium diphenyltetrazolium bromide for (MTT) assay. There was a statistically significant difference between ABCB5 mAb-treated and isotype control mAb-treated cells as determined by analysis of variance (ANOVA), (F(1, 144)=48.42, P<0.0001). A multiple comparisons test showed that anti-ABCB5 mAb inhibited the proliferation of GBM cells and that this inhibition was statistically significant at concentrations ≥20 μg/ml (concentration, adjusted P-value: 20, 0.0293; 50, 0.0021; 100, <0.0001; 200, <0.0001) (FIG. 3A). Furthermore, antibody-mediated ABCB5 blockade led to increased apoptosis of GBM cells. LN-229, LN-18, and U-87 MG cells incubated with 100 μg/ml ABCB5 mAb vs. isotype-matched control antibody for 72 hours showed a 2.3-fold increase in the percentage of apoptotic cells (early+late) (ABCB5 mAb vs. isotype control: 23.90±3.5% vs. 10.55±1.6%, P<0.01, mean±SEM) (FIG. 3B) as determined by dual color flow cytometry using Annexin-V (APC, FL4 fluorescence) and propidium iodide (FL2 fluorescence) staining. These results suggest that, similar to its role in normal stem cells, ABCB5 serves as a growth-inducing and anti-apoptotic molecule in GBM.

To evaluate the potential role of ABCB5 in GBM growth in vivo, the effect of ABCB5 blockade on tumor growth in an established human-to-mouse GBM xenotransplantation model was tested (Singh, S. K., et al., (2004) Identification of human brain tumour initiating cells. Nature 432, 396-401). In this experiment, LN-229 and U-87 MG GBM cell lines were employed, which exhibited differential CD133-positive GBM stem cell response to ABCB5 blockade in vitro, to test whether this in vitro response translates into an in vivo effect on tumor growth. The LN-18 cell line was excluded from the in vivo study as, consistent with published reports, it reproducibly failed to form tumors. LN-229 and U-87 MG GBM cells were injected subcutaneously into immunodeficient NSG mice as described (Singh, S. K., et al., (2004) Identification of human brain tumour initiating cells. Nature 432, 396-401). Examination of tumor xenografts revealed reduced tumor growth over time after functional blockade of ABCB5 in mice injected with LN-229 GBM cells (FIG. 3C, left). A statistically significant reduction in endpoint tumor volume was observed for LN-229 (ABCB5 mAb vs. isotype control: 158.1±22.26 mm³ vs. 68.13±14.75 mm³, P=0.0098, mean±SEM) but not U-87 MG GBM cells (ABCB5 mAb vs. isotype control: 473.3±156.5 mm³ vs. 460.4±111.7 mm³, P=0.95, mean±SEM) by unpaired t-test. Tumor weight was also decreased in mice treated with anti-ABCB5 mAb compared to isotype control for LN-229 but not U-87 MG GBM cells (ABCB5 mAb vs. isotype control: LN-229 day 31: 0.315 gm±0.043 gm vs. 0.181 gm±0.032 gm, P=0.0372; U-87 MG day 24 (mice sacrificed early due to tumor burden): 0.75 gm±0.23 gm vs. 0.724 gm±0.149 gm, P=0.926, mean±SEM) (FIG. 3C, right). These results suggest that GBM tumors might exhibit differential response to mAb-mediated ABCB5 blockade based on their genetic inter-tumor heterogeneity and divergent growth kinetics.

The growth inhibitory and pro-apoptotic effects of ABCB5 blockade in LN-229 tumors were further validated by assessing expression of the proliferation marker Ki-67 and apoptosis marker cleaved caspase-3 in tumor xenografts of ABCB5 mAb vs. isotype control-treated mice. Immunohistochemical analysis (FIG. 3D) of ABCB5 mAb-treated tumor xenografts showed a 2.0-fold decrease in Ki-67 (ABCB5 mAb vs. isotype control: 22.1±3.1% vs. 43.3±3.2%, P=0.003, mean±SEM) (FIG. 3E, left) and a 3.6-fold increase in cleaved caspase-3 (ABCB5 mAb vs. isotype control: 4.0±0.4% vs. 1.1±0.3%, P=0.0008, mean±SEM) (FIG. 3E, right) positive nuclei, underscoring the tumorigenic and anti-apoptotic role of ABCB5 in this GBM model system.

### Example 4: Targeted ABCB5 Blockade Augments TMZ-Mediated Inhibition of GBM Cell Proliferation and Promotes Drug-Induced Apoptosis In Vitro and In Vivo

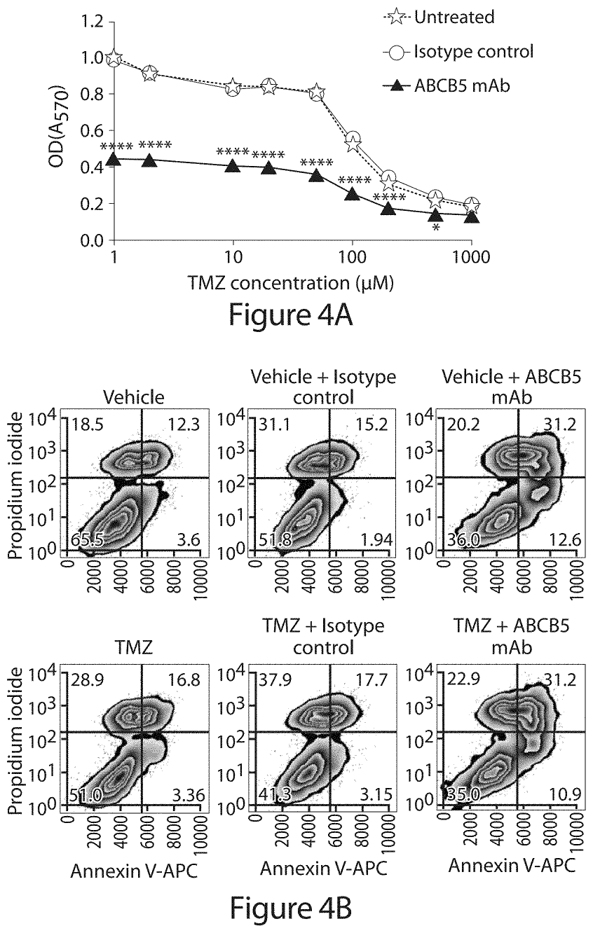

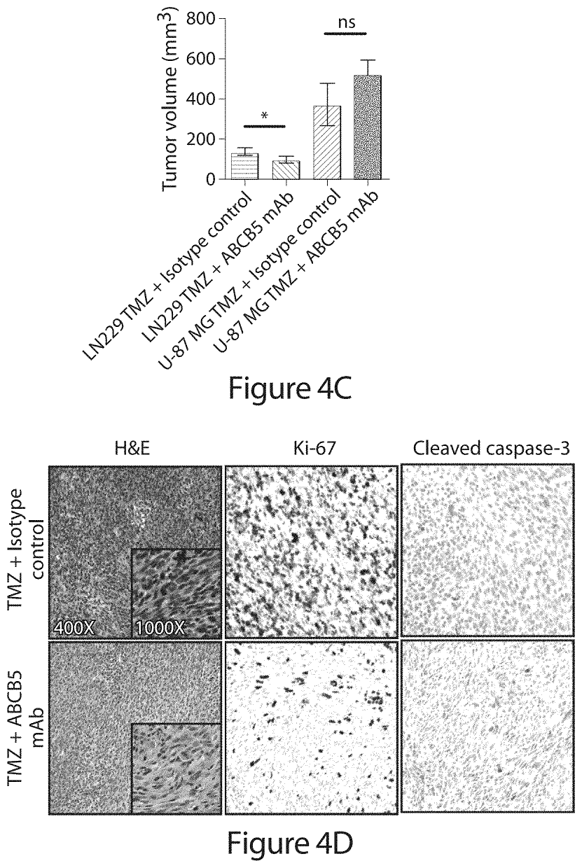

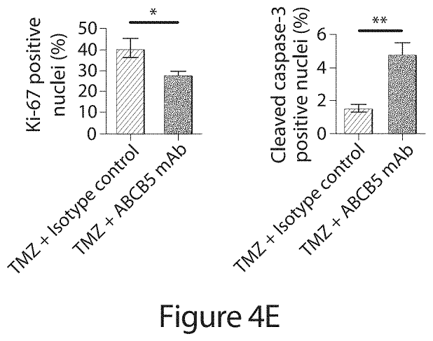

To further dissect a potential role of ABCB5 in GBM therapeutic resistance, we subjected GBM cell cultures to TMZ treatment in combination with mAb-mediated ABCB5 blockade. LN-229, LN-18, and U-87 MG cells were pre-incubated for 2 hours with 100 μg/ml ABCB5 mAb or isotype control mAb, and then treated with TMZ (0-1000 μM) for 72 hours. A statistically significant reduction in cell proliferation as measured by MTT assay was observed by ANOVA (F(1,160)=1756, P<0.0001) and a multiple comparisons test showed that the inhibitory effect of TMZ on proliferation of GBM cells was further augmented by antibody-mediated blockade of ABCB5 and that this inhibitory effect was statistically significant at all timepoints measured with the exception of the highest concentration of TMZ (1000 μM) (adjusted P-value <0.0001 for concentrations ≤200 μM, P=0.0101 at 500 μM) (FIG. 4A). Targeted inhibition of ABCB5 also augmented TMZ-induced apoptosis of GBM cells as determined by dual color flow cytometry using Annexin-V (APC, FL4 fluorescence) and propidium iodide (FL2 fluorescence) staining. GBM cells pre-incubated for 2 hours with 100 μg/ml ABCB5 mAb followed by treatment with 100 μM TMZ for 72 hours showed an increased percentage (2.3-fold) of apoptotic cells (early+late) compared to those incubated with isotype control mAb (ABCB5 mAb vs. isotype control: 43.5±0.7% vs. 18.6±1.2%, P<0.0001, mean±SEM) (FIG. 4B), suggesting that ABCB5 might serve as a mediator of TMZ resistance in human GBM.

To test whether mAb-mediated ABCB5 blockade led to sensitization of established GBM tumors in vivo, we subjected LN-229 and U-87 MG xenografted NSG mice to TMZ therapy in the presence of ABCB5 mAb or isotype control. Mice xenografted with LN-229 cells exhibited a 1.4-fold decrease in tumor volume after treatment with TMZ in the presence of ABCB5 mAb compared to mice that received the drug in the presence of isotype-matched control mAb (ABCB5 mAb vs. isotype control: 98.50±20.34 mm³ vs. 138.6±19.67 mm³, P=0.025, mean±SEM). Mice xenografted with U-87 MG cells exhibited no significant difference in tumor volume after treatment with TMZ in the presence of ABCB5 mAb compared to mice that received the drug in the presence of isotype-matched control mAb (ABCB5 mAb vs. isotype control: 521.7±75.52 mm³ vs. 372.3±107.4 mm³, P=0.059, mean±SEM) (FIG. 4C). Tumors from mice injected with LN-229 cells treated with TMZ in the presence of ABCB5 mAb exhibited a 1.5-fold decrease in Ki-67 (ABCB5 mAb vs. isotype control: 27.8±2.1% vs. 40.9±4.6%, P=0.03, mean±SEM) and 3.0-fold increase in cleaved caspase-3 (ABCB5 mAb vs. isotype control: 4.8±0.6% vs. 1.6±0.2%, P=0.0013, mean±SEM) (FIGS. 4D & E) positive nuclei compared to isotype control mAb treated mice. These findings underline the potential role of ABCB5 targeting in the reversal of GBM therapeutic resistance to TMZ and also highlight differential response of GBM tumors to mAb-mediated ABCB5 blockade based on tumor molecular subtype and differences in growth kinetics.

### Example 5: Antibody-Mediated Blockade of ABCB5 Releases GBM Cells from TMZ-Induced G2/M Arrest

To examine potential molecular mechanisms responsible for the attenuation of GBM tumor growth in the presence of TMZ and mAb-mediated ABCB5 blockade, we performed microarray gene expression analyses of ABCB5-positive and ABCB5-negative cells sorted by flow cytometry from U-87 MG, LN-18, and LN-229 GBM cell lines. Principal component analysis (PCA) performed on all genes detected by microarray showed separation specific to the cell lines on PC1 and PC2 while PC3 separated the ABCB5-positive and ABCB5-negative cells (FIG. 5A). We generated a list of 1661 genes differentially expressed (P<0.05) between the ABCB5-positive and ABCB5-negative cells in all three cell lines and used this list as input into Ingenuity Pathway Analysis (IPA). IPA determined 489 disease and functional categories to be enriched between the ABCB5-positive and ABCB5-negative cells. Strikingly, 13.7% of these total categories were related to cell cycle (as compared to 5.9% for cancer and 7.6% for neuro/nervous system) (FIG. 5B). Specific enriched categories (reported as -log₁₀(p-value), included Cancer, Cell Death and Survival (5.41), Cell Cycle: Aneuploidy (3.22), Cell Cycle: Spindle Checkpoint (2.36), Cell Cycle: G2/M Transition (2.28), Cell Cycle: Interphase (2.73), Cell Cycle: Telomere Capping (2.22), Cell Cycle Progression (2.19), and Cell Cycle: M Phase (2.15) (FIG. 5C).

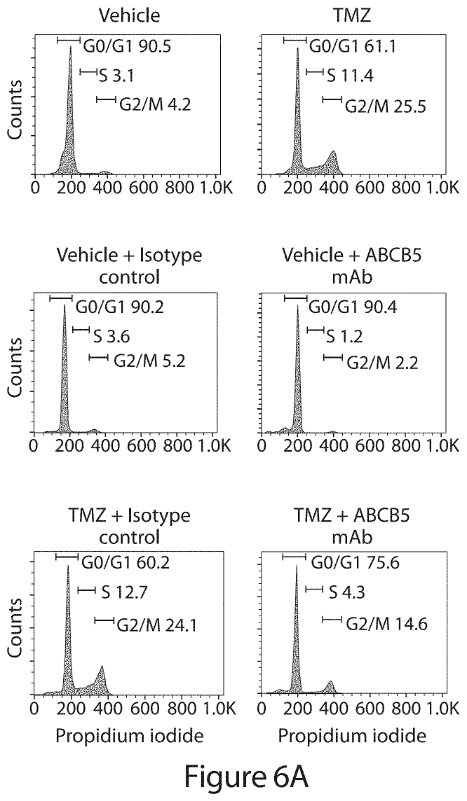

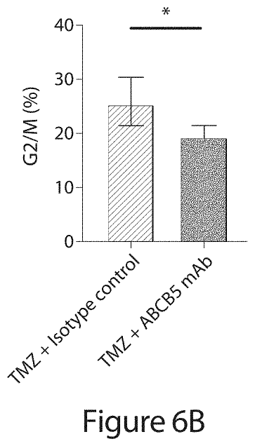

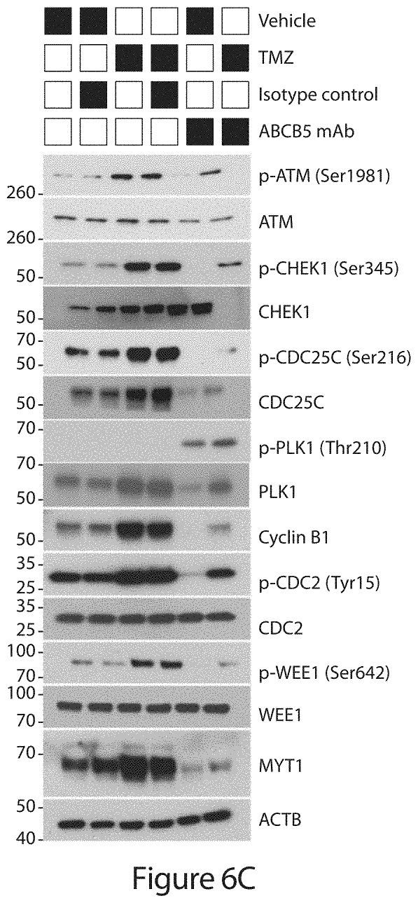

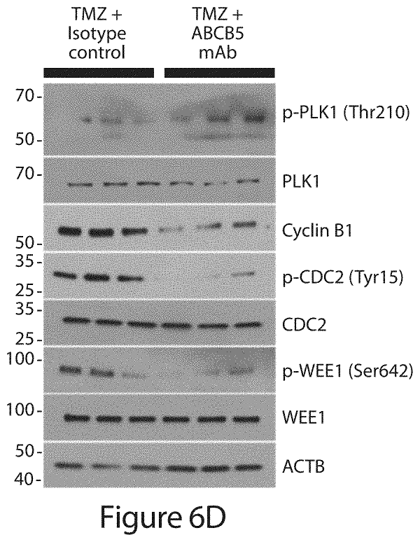

Based on these results, we examined whether ABCB5 blockade could reverse GBM chemoresistance through modulation of the G2/M checkpoint regulators and subsequent reversal of drug-induced G2/M arrest. LN-229, LN-18, and U-87 MG cells were pre-incubated for 2 hours with 100 μg/ml ABCB5 mAb or isotype control mAb followed by treatment with 100 μM TMZ for 72 hours. Cells were fixed in ice-cold ethanol, stained in propidium iodide/RNase buffer, and DNA content was analyzed by flow cytometry following FL2H versus FL2W analysis for doublet elimination (FIG. 6A). We found that antibody-mediated functional inhibition of ABCB5 is capable of abrogating TMZ-induced G2/M arrest in GBM cell cultures, as evidenced by the 1.4-fold reduced cell accumulation in G2/M phase of cell cycle following ABCB5 mAb and TMZ treatment, compared to cells that received TMZ treatment in the presence of isotype control (ABCB5 mAb vs. isotype control: 18.9±2.5% vs. 25.76±4.6%, P=0.0225, mean±SEM) (FIG. 6B). Western blot analysis revealed that ABCB5 blockade reversed TMZ-mediated G2/M arrest by inhibiting cell cycle arrest-inducing checkpoint molecules (ATM, CHK1, WEE1 and MYT1), and augmenting the activation of molecules that drive cells from G2 phase to mitosis by either inducing their phosphorylation (as for PLK1) or by removing their inhibitory phosphorylation (as for CDC25C and CDC2) (FIG. 6C). In support of these in vitro findings, similar changes in cell cycle protein expression were observed in tumor xenografts from mice treated with TMZ in the presence of either ABCB5 mAb or isotype control mAb (FIG. 6D).

### Example 6: Knockdown of ABCB5 Mimics Growth-Inhibition of ABCB5 Blockade and Releases GBM Cells from TMZ-Induced G2/M Arrest

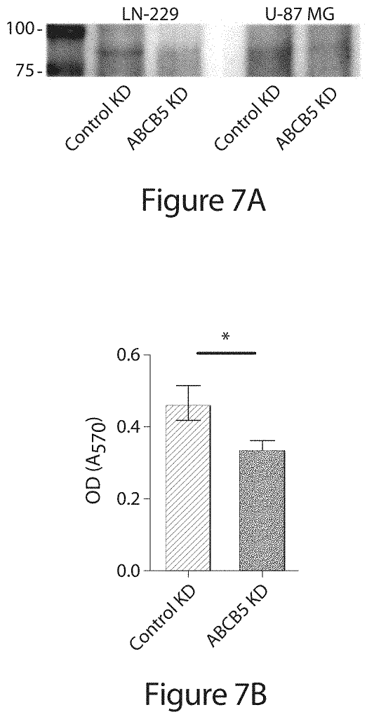

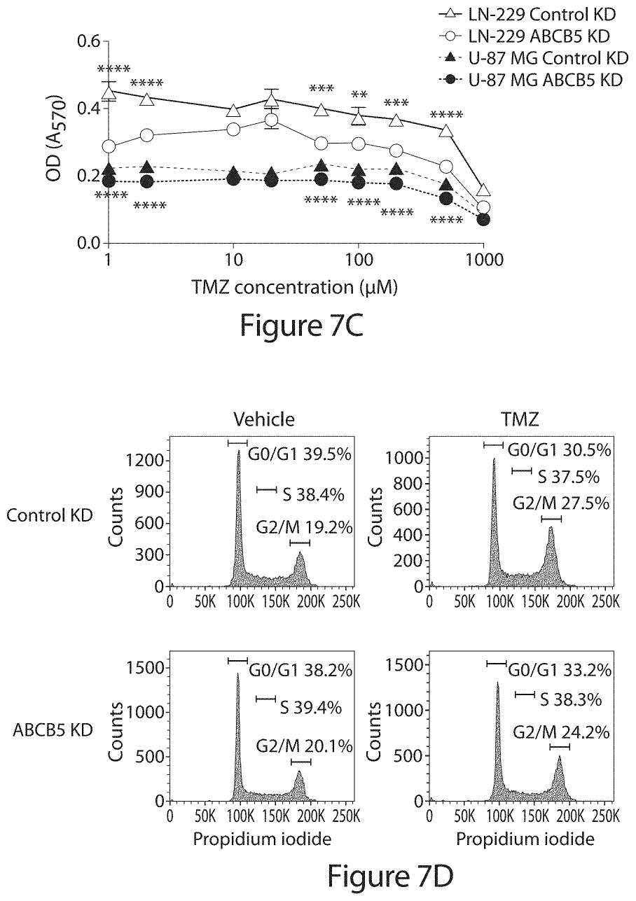

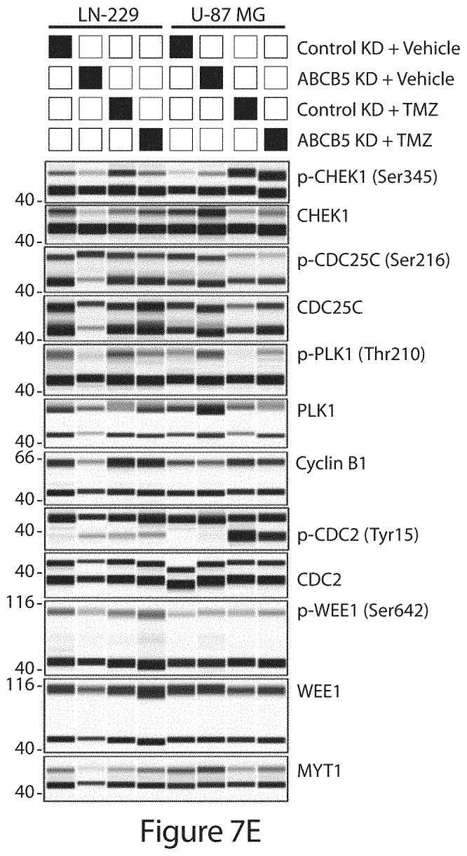

Knockdown (KD) of ABCB5 by short hairpin RNA (shRNA) was confirmed by immunoprecipitation (IP)-Western blot. LN-229 and U-87 MG GBM cells transfected with ABCB5-targeting shRNA showed reduction of ABCB5 protein expression compared to their respective Control KD cell variants (LN-229 Control KD vs. ABCB5 KD: 3.83×10⁸ vs. 2.15×10⁸ integrated density; U-87 MG Control KD vs. ABCB5 KD: 4.10×10⁸ vs. 3.34×10⁸ integrated density) (FIG. 7A). Similar to antibody-mediated ABCB5 blockade, ABCB5 KD decreased proliferation of LN-229 and U-87 MG GBM compared to their respective Control KD cell variants as measured by MTT assay (Control KD vs. ABCB5 KD: 0.47+0.05 OD₅₇₀ vs. 0.34±0.03 OD₅₇₀, P=0.0406, mean±SEM) (FIG. 7B). Treatment of ABCB5 KD and Control KD cell lines with TMZ (0-1000 μM) showed a statistically significant reduction in cell proliferation as measured by MTT assay and observed by ANOVA for LN-229 (F(1,120)=197.2, P<0.0001) and a multiple comparisons test showed that the inhibitory effect of TMZ on proliferation of GBM cells was further augmented by antibody-mediated blockade of ABCB5 and that this inhibitory effect was statistically significant at all timepoints measured with the exception of 10 μM, 20 μM, and the highest concentration of TMZ of 1000 μM (adjusted P-value <0.0001 for 0-2 μM and 500 μM, P=0.0002 at 50 μM, P=0.0041 at 100 μM, P=0.0008 at 200 μM). This effect was similar for U-87 MG, ANOVA F(1,119)=193.3, P<0.0001), with corresponding results for the multiple comparisons test (adjusted P-value <0.0001 for 1-2 μM and 50-500 μM, P=0.0001 at 0 μM) (FIG. 7C).

Flow cytometry and Western blotting were used to determine if KD of ABCB5 releases GBM cells from G2/M arrest and modulates G2/M checkpoint regulators. LN-229 ABCB5 KD and Control KD GBM cells were treated with 100 μM TMZ for 72 hours. Cells were fixed in ice-cold ethanol, stained with propidium iodide/RNase buffer, and DNA content was analyzed by flow cytometry following FL2H versus FL2W analysis for doublet elimination. ABCB5 KD is capable of mitigating TMZ-induced G2/M arrest in GBM cell cultures, as evidenced by the reduction in cell accumulation in G2/M phase of cell cycle following TMZ treatment compared to Control KD cells that received TMZ (Control KD vs. ABCB5 KD: 27.5% vs. 24.2%) (FIG. 7D). Similar to the mAb-mediated ABCB5 blockade, shRNA-mediated ABCB5 knockdown (KD) reduced TMZ-induced inhibitory CHEK1 phosphorylation and inhibitory Cyclin B1 expression in LN229 and U-87 MG cells, reduced TMZ-induced inhibitory CDC25 phosphorylation in LN229 cells, and also reduced TMZ-induced inhibitory CDC2 phosphorylation and induced PLK1 phosphorylation in U-87 MG cells (FIG. 7E).

In the current study, the use of ABCB5 as a novel therapeutic target in GBM was examined. The results revealed ABCB5 expression in primary human GBM tumors and three established GBM cell lines. Using mAb-based ABCB5 inhibition strategies in vitro or ABCB5 blockade in tumor xenotransplantation models in vivo, we show for the first time that targeting ABCB5 can significantly inhibit tumor growth and sensitize a TP53-mutant PTEN-wild-type GBM subtype to TMZ treatment. We demonstrate that ABCB5 inhibition results in modulation of the G2/M checkpoint regulators and subsequent reversal of TMZ-induced G2/M arrest.

The finding of specific ABCB5 overexpression in human GBM compared to less aggressive brain tumors and the significant correlation of ABCB5 expression with OS among GBM patients points to a potential role of ABCB5 as a determinant of GBM aggression and therapeutic resistance. This is further supported by the observed expression of ABCB5 on CD133-positive GBM CSCs and a significant positive correlation between CD133 and ABCB5 expression in clinical GBM specimens. CD133-positive GBM subpopulations are enriched for CSCs and exhibit higher rates of self-renewal, proliferation, and tumorigenicity compared to CD133-negative populations. Moreover, enrichment of CD133-positive CSCs is observed in GBM cultures, xenografts, and clinical tumor specimens following radiation and chemotherapy, highlighting their role in GBM progression and therapy resistance. Our data indicate that, similar to its function in normal tissue stem cells, ABCB5 contributes significantly to the survival of GBM CSCs, since antibody-mediated ABCB5 blockade leads to a significant decline in the CD133-positive CSC subpopulation in human GBM cell lines. Our finding that ABCB5 blockade can attenuate proliferation and promote apoptosis underscores the potential role of ABCB5 targeting in the reversal of CSC-mediated GBM tumorigenesis.

All references cited herein are fully incorporated by reference. Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

1. A method for treating Glioblastoma multiforme (GBM), comprising
administering to a subject having GBM an inhibitor of ATP-binding cassette subfamily B member 5 (ABCB5) and a chemotherapeutic agent, wherein the chemotherapeutic agent is an alkylating agent in an effective amount to treat the GBM.

2. The method of claim 1, wherein the alkylating agent is Temozolomide.

3. A method for treating a chemoresistant cancer, comprising:
identifying a subject having a chemoresistant cancer, administering an effective amount of an ABCB5 inhibitor to reverse a chemotherapy induced G2/M arrest in cancer cells of the subject and administering a chemotherapeutic agent to the subject to promote cancer cell death.

4. A method for treating a cancer in a subject, wherein the cancer is associated with G2/M cell cycle arrest, comprising:
identifying the cancer as a cancer having cells in G2/M cell cycle arrest and administering an effective amount of an ABCB5 inhibitor to the subject to reverse G2/M cell cycle arrest in the cells.

5. The method of claim 4, further comprising administering a chemotherapeutic agent to the subject to promote cancer cell death.

6. The method of claim 3, wherein the chemotherapeutic agent is an alkylating agent.

7. The method of claim 6, wherein the alkylating agent is Temozolomide.

8. The method of claim 1, wherein the subject is identified as having an ABCB5+ cancer.

9. The method of claim 1, wherein the inhibitor of ABCB5 is an anti-ABCB5 antibody or fragment thereof.

10. The method of claim 9, wherein the anti-ABCB5 antibody or fragment thereof has specificity for a cyclical form or a linear form of an extracellular polypeptide of the protein.

11. The method of claim 9, wherein the anti-ABCB5 antibody or fragment thereof alters the conformation of an ABCB5 PIP2 binding site.

12. The method of claim 9, wherein the anti-ABCB5 antibody is a monoclonal antibody.

13. The method of claim 1, wherein the inhibitor of ABCB5 is a PIP2 antagonist.

14. The method of claim 1, wherein the inhibitor of ABCB5 is a lipid analog.

15. The method of claim 1, wherein the inhibitor of ABCB5 is an inhibitory nucleic acid.

16. The method of claim 1, wherein the inhibitor of ABCB5 is an enzyme.

17. The method of claim 1, wherein the inhibitor of ABCB5 is anti-receptor tyrosine kinase (RTK) antibody.

18. The method of claim 1, wherein the inhibitor of ABCB5 is an inhibitor of insulin receptor (InsR).

19. The method of claim 1, wherein the inhibitor of ABCB5 is an inhibitor of insulin-like growth factor-1 receptor (IGF1R).

20. The method of claim 1, wherein the inhibitor of ABCB5 is an inhibitor of signal integrating adapter molecule (IRS-1).

21. A method for identifying a Glioblastoma multiforme (GBM) as a chemoresistant cancer, comprising:
isolating a GBM cancer cell from a subject and performing an assay to determine whether the GBM cancer cell expresses ABCB5, wherein if the GBM cancer cell expresses ABCB5 the GBM is a chemoresistant cancer.

22. A bispecific antibody, comprising two antigen binding domains, wherein a first antigen binding domain is specific for ABCB5 and a second antigen binding domain is specific for a receptor tyrosine kinase (RTK).

23. The bispecific antibody of claim 22, wherein a level of occupancy of an antigen is higher using the bispecific antibody as compared to the level of occupancy using an antibody monospecific for the antigen.

24. The bispecific antibody of claim 22, wherein the first antigen binding region comprises a single chain variable fragment (scFv).

25. The bispecific antibody of claim 22, wherein binding of the bispecific antibody to the target cell results in downregulation of the signaling pathway.

26. The bispecific antibody of claim 22, wherein an IC50 value of the bispecific antibody is decreased at least 100-fold as compared to an IC50 value of an antibody monospecific to the antigen.
